SEQUENCE LISTING (15 sequences)

SEQ ID NO: 1 - DNA, 407 bp, T. reesei
atgtctgagacgcacacctacgagttcaacgtcaccatgacctgcggcggctgctccggcgccatcgaccgagtcctcaagaagctcgagggtacgttcttgaacaatcattctcccctctcctctcctctcctcccctctctctctccctctctctcctccgtcatgccgtaggagcactgtctgcgcccctccccctccaaagaaaaacacagcactgactcggttggttttctttctttctcgcaggcgtcgaaagctacgaagtctccctcgacaaccagaccgcaaaggtcgtcaccgcgctgccctacgagacggtcctgaccaagattgccaagacgggcaagaagatcaactcggcgacggccgacggcgtgccgcagtctgtcgaggtatctgtgtag

SEQ ID NO: 2 - DNA, 251 bp, T. reesei
atgtctgagacgcacacctacgagttcaacgtcaccatgacctgcggcggctgctccggcgccatcgaccgagtcctcaagaagctcgagggggcgtcgaaagctacgaagtctccctcgacaaccagaccgcaaaggtcgtcaccgcgctgccctacgagacggtcctgaccaagattgccaagacgggcaagaagatcaactcggcgacggccgacggcgtgccgcagtctgtcgaggtatctgtgtag

SEQ ID NO: 3 - protein, 82 aa, T. reesei
MSETHTYEFNVTMTCGGCSGAIDRVLKKLEGVESYEVSLDNQTAKVVTALPYETVLTKIAKTGKKINSATADGVPQSVEVSV

SEQ ID NO: 4 - DNA, 3605 bp, T. reesei
atggccccaacatacatcaaagtccccgggcgggacaatgatgagcatgcgagtgcgacccttacgccaaagagcgcgcacatggccacaaccactctgcgcgttggtggcatgacgtaggtttcgtccgtttccggctgtgcttccggccaaggtctgcagcacaagcatggctggtcattctttctaacacttcttcttgcagatgtggttcgtgcacagcagccgtcgagggcggcttcaagggcgtcaagggcgttggtaccgtctccgtcagccttgttatggagagggctgtcgtaatgcacgacccccggatcatcagcgctgaacaggttcgagagattatcgaagattgtggattcgacgctgagctgctgtcgacggacctcttgagcccactcgtccctcgattctcggatgccaagggggatgaggacatcgatagcggcctcttgacgaccacggtagccatcgaaggcatgacgtgtggcgcctgtacatctgctgtcgagggtggattcaaggatatcccaggtgtcaagagcttcagcatctcgcttctttctgagcgagccgtcatcgaacacgatccagaacttttgcccaccgacaagattaccgaaatcatcgaagaccggggctttggtgccgaaatcgtcgattccgtgaaggcgcaacctggcagcagtaccgaggctgagaacccagcaagtcatgtcgtgactacgacggtagccatcgaaggaatgacttgcggtgcctgtacgtctgctgttgagggaggctttcagggagttgacggcatcctgaaattcaacatcagtcttctggccgaaagggcagtcattactcacgatgtcaccaagatctccgccgaacagatttccgaaatcgttgaagaccggggatttggtgctacggttttgtccaccgtcccggaggcaaacgatctcagcagtacgacctcgcagttcaaaatctatggcagcccggacgccgccactgcaaaggagctggaggaaaagctgctggcacttgctggtgttaaatctgcttccctcagcctatcaacggaccgcctgtccgtcacgcaccagcctgccgtcattgggctccgagggatcgtcgaggcggtagaggcgcaaggcctgaatgctttggtggcggacagccacgacaacaacgcgcaactcgaatccttggccaagactcgcgagatccaggaatggaggacggcgtgcaagacgtccgcctcgttcgccattccggtattcgttctttccatggtgttgcctatgatctcagacagtctgaacctgagtctaatccaccttggccatggtctctacctcggcgacgtcgtcaacttggtactcacaacacctgttcagtttggggttggaaagcgcttttacgtctcggccttcaagtcgctcaagcaccgttcgccgactatggatgtgctcgtcatgctcggcacctcctgcgcttacttcttcagcatcttctccatggtcatctctatcctcttcgagcctcattccccgccgggcacgatctttgacaccagcaccatgctcatcacctttgtgaccttgggccgctatcttgagaacagcgccaagggtcagacatcaaaggctctgtcccgtctcatgtctctagccccgtcgatggccaccatctacacggatcccattgccgcggagaaggcagcagaatcatgggccaagtcaaccgatacacccgcggatgcgaaaggccaaccgtctggagatgcgagcggctcgtcgtacgaggagaagagcatccctactgagctgcttcaggtgggagatatcgtcgtcatccgacccggtgataagattccggcggacggcgtcgttatgcgaggagagacctacgtcgacgagagcatggtcaccggagaggcaatgccggtgcagaagaggattggcagcaacgtgattggaggcacggtcaacggcaacggcagagtggactttcgcgtcacccgagccgggcgggatacccagctcagtcagattgtcaagcttgttcaggacgcgcagacgacgagggcgcctattcaaaaggtggccgacactttggctggctactttgtgcctacaatcttgctgctcggcatcctcaccttccttggctggttgatcctcagccacgccctgtcgcacccccctatgattttcttgaagaacaccagtggtggcaaggtcatgatttgcgtcaagctgtgcatctccgtcattgtatttgcatgcccttgtgctctgggcctggccacgccgacagctgtcatggtaggcacgggcgtgggcgctgagaatggcatcctcatcaaaggcggagctgcgctggagcgaaccacccaggttaccaaagtcgtcttggacaaaaccggcacaatcactcgtggcaaaatggaggtcgccaagagcggccttgtgtttccctggaatgacaacgtgtcgcagaccaaagtctggtgggccgctgtcggtctggcggaaatgggcagcgagcaccctatcggaagggcgattctggcagcggccaaggcagaagtcggcatccttgaagccgaagccgccattccaggaagcgtcaatgatttcaagttgactgttggcaagggcatcgatgctatcgttgaacctgcattatccggtgatcggacacgctatagggtccttgctggaaatgtcaccttccttgaagagaacggcgtcgaggtccccaaggatgccgtcgaggcagcagagcgaatcaactcgtccgtcaagagctcacgagccaaggctgtgactgcgggcacgaccaacatctttgtcgccattgatggaaagtacagcggccacctttgtctctccgacaccatcaaagatggggcggccggggtcatttctgtactgcatagcatgggcatcaagacggccatggtgacgggagaccagcgacccaccgccctggccgttgccgccctcgtgggcatctctcccgaggacgtgtttgccggcgtcagccccgaccagaagcaggtgatagtacagcagttccagaaccagggagaggtggtcgccatggtgggagacggcatcaacgactcgccggccctcgctacggccgacgttggtatcgccatgtcgagcggaacggacgtggccatggaggccgcagatgttgtgcttatgcgtcccgacgacctgctgagcatcccgtccgccatccacctcactcggaccatcttccgccgcatcaagctgaacctggcgtgggcatgcatctacaacattgtcggcctgcccattgccatgggtttcttcctgccgtttggcatccacatgcaccccatgttcgccgggttcgccatggcctgcagtagcattagtgtagtggttagcagcctggcgctccgatggtggcaacgaccgcagtggatggacgaggcgtccgaaccggcgggtggcctgcgctggatgagcggcacgggcatcgttggctgggctaaggagacgtttggacgcgtcaggagagggaagcgtgaggagggttacgtggcgttggagaatttagaggtctga

SEQ ID NO: 5 - DNA, 3516 bp, T. reesei
atggccccaacatacatcaaagtccccgggcgggacaatgatgagcatgcgagtgcgacccttacgccaaagagcgcgcacatggccacaaccactctgcgcgttggtggcatgacatgtggttcgtgcacagcagccgtcgagggcggcttcaagggcgtcaagggcgttggtaccgtctccgtcagccttgttatggagagggctgtcgtaatgcacgacccccggatcatcagcgctgaacaggttcgagagattatcgaagattgtggattcgacgctgagctgctgtcgacggacctcttgagcccactcgtccctcgattctcggatgccaagggggatgaggacatcgatagcggcctcttgacgaccacggtagccatcgaaggcatgacgtgtggcgcctgtacatctgctgtcgagggtggattcaaggatatcccaggtgtcaagagcttcagcatctcgcttctttctgagcgagccgtcatcgaacacgatccagaacttttgcccaccgacaagattaccgaaatcatcgaagaccggggctttggtgccgaaatcgtcgattccgtgaaggcgcaacctggcagcagtaccgaggctgagaacccagcaagtcatgtcgtgactacgacggtagccatcgaaggaatgacttgcggtgcctgtacgtctgctgttgagggaggctttcagggagttgacggcatcctgaaattcaacatcagtcttctggccgaaagggcagtcattactcacgatgtcaccaagatctccgccgaacagatttccgaaatcgttgaagaccggggatttggtgctacggttttgtccaccgtcccggaggcaaacgatctcagcagtacgacctcgcagttcaaaatctatggcagcccggacgccgccactgcaaaggagctggaggaaaagctgctggcacttgctggtgttaaatctgcttccctcagcctatcaacggaccgcctgtccgtcacgcaccagcctgccgtcattgggctccgagggatcgtcgaggcggtagaggcgcaaggcctgaatgctttggtggcggacagccacgacaacaacgcgcaactcgaatccttggccaagactcgcgagatccaggaatggaggacggcgtgcaagacgtccgcctcgttcgccattccggtattcgttctttccatggtgttgcctatgatctcagacagtctgaacctgagtctaatccaccttggccatggtctctacctcggcgacgtcgtcaacttggtactcacaacacctgttcagtttggggttggaaagcgcttttacgtctcggccttcaagtcgctcaagcaccgttcgccgactatggatgtgctcgtcatgctcggcacctcctgcgcttacttcttcagcatcttctccatggtcatctctatcctcttcgagcctcattccccgccgggcacgatctttgacaccagcaccatgctcatcacctttgtgaccttgggccgctatcttgagaacagcgccaagggtcagacatcaaaggctctgtcccgtctcatgtctctagccccgtcgatggccaccatctacacggatcccattgccgcggagaaggcagcagaatcatgggccaagtcaaccgatacacccgcggatgcgaaaggccaaccgtctggagatgcgagcggctcgtcgtacgaggagaagagcatccctactgagctgcttcaggtgggagatatcgtcgtcatccgacccggtgataagattccggcggacggcgtcgttatgcgaggagagacctacgtcgacgagagcatggtcaccggagaggcaatgccggtgcagaagaggattggcagcaacgtgattggaggcacggtcaacggcaacggcagagtggactttcgcgtcacccgagccgggcgggatacccagctcagtcagattgtcaagcttgttcaggacgcgcagacgacgagggcgcctattcaaaaggtggccgacactttggctggctactttgtgcctacaatcttgctgctcggcatcctcaccttccttggctggttgatcctcagccacgccctgtcgcacccccctatgattttcttgaagaacaccagtggtggcaaggtcatgatttgcgtcaagctgtgcatctccgtcattgtatttgcatgcccttgtgctctgggcctggccacgccgacagctgtcatggtaggcacgggcgtgggcgctgagaatggcatcctcatcaaaggcggagctgcgctggagcgaaccacccaggttaccaaagtcgtcttggacaaaaccggcacaatcactcgtggcaaaatggaggtcgccaagagcggccttgtgtttccctggaatgacaacgtgtcgcagaccaaagtctggtgggccgctgtcggtctggcggaaatgggcagcgagcaccctatcggaagggcgattctggcagcggccaaggcagaagtcggcatccttgaagccgaagccgccattccaggaagcgtcaatgatttcaagttgactgttggcaagggcatcgatgctatcgttgaacctgcattatccggtgatcggacacgctatagggtccttgctggaaatgtcaccttccttgaagagaacggcgtcgaggtccccaaggatgccgtcgaggcagcagagcgaatcaactcgtccgtcaagagctcacgagccaaggctgtgactgcgggcacgaccaacatctttgtcgccattgatggaaagtacagcggccacctttgtctctccgacaccatcaaagatggggcggccggggtcatttctgtactgcatagcatgggcatcaagacggccatggtgacgggagaccagcgacccaccgccctggccgttgccgccctcgtgggcatctctcccgaggacgtgtttgccggcgtcagccccgaccagaagcaggtgatagtacagcagttccagaaccagggagaggtggtcgccatggtgggagacggcatcaacgactcgccggccctcgctacggccgacgttggtatcgccatgtcgagcggaacggacgtggccatggaggccgcagatgttgtgcttatgcgtcccgacgacctgctgagcatcccgtccgccatccacctcactcggaccatcttccgccgcatcaagctgaacctggcgtgggcatgcatctacaacattgtcggcctgcccattgccatgggtttcttcctgccgtttggcatccacatgcaccccatgttcgccgggttcgccatggcctgcagtagcattagtgtagtggttagcagcctggcgctccgatggtggcaacgaccgcagtggatggacgaggcgtccgaaccggcgggtggcctgcgctggatgagcggcacgggcatcgttggctgggctaaggagacgtttggacgcgtcaggagagggaagcgtgaggagggttacgtggcgttggagaatttagaggtctga

SEQ ID NO: 6 - protein, 1171 aa, T. reesei
MAPTYIKVPGRDNDEHASATLTPKSAHMATTTLRVGGMTCGSCTAAVEGGFKGVKGVGTVSVSLVMERAVVMHDPRIISAEQVREIIEDCGFDAELLSTDLLSPLVPRFSDAKGDEDIDSGLLTTTVAIEGMTCGACTSAVEGGFKDIPGVKSFSISLLSERAVIEHDPELLPTDKITEIIEDRGFGAEIVDSVKAQPGSSTEAENPASHVVTTTVAIEGMTCGACTSAVEGGFQGVDGILKFNISLLAERAVITHDVTKISAEQISEIVEDRGFGATVLSTVPEANDLSSTTSQFKIYGSPDAATAKELEEKLLALAGVKSASLSLSTDRLSVTHQPAVIGLRGIVEAVEAQGLNALVADSHDNNAQLESLAKTREIQEWRTACKTSASFAIPVFVLSMVLPMISDSLNLSLIHLGHGLYLGDVVNLVLTTPVQFGVGKRFYVSAFKSLKHRSPTMDVLVMLGTSCAYFFSIFSMVISILFEPHSPPGTIFDTSTMLITFVTLGRYLENSAKGQTSKALSRLMSLAPSMATIYTDPIAAEKAAESWAKSTDTPADAKGQPSGDASGSSYEEKSIPTELLQVGDIVVIRPGDKIPADGVVMRGETYVDESMVTGEAMPVQKRIGSNVIGGTVNGNGRVDFRVTRAGRDTQLSQIVKLVQDAQTTRAPIQKVADTLAGYFVPTILLLGILTFLGWLILSHALSHPPMIFLKNTSGGKVMICVKLCISVIVFACPCALGLATPTAVMVGTGVGAENGILIKGGAALERTTQVTKVVLDKTGTITRGKMEVAKSGLVFPWNDNVSQTKVWWAAVGLAEMGSEHPIGRAILAAAKAEVGILEAEAAIPGSVNDFKLTVGKGIDAIVEPALSGDRTRYRVLAGNVTFLEENGVEVPKDAVEAAERINSSVKSSRAKAVTAGTTNIFVAIDGKYSGHLCLSDTIKDGAAGVISVLHSMGIKTAMVTGDQRPTALAVAALVGISPEDVFAGVSPDQKQVIVQQFQNQGEVVAMVGDGINDSPALATADVGIAMSSGTDVAMEAADVVLMRPDDLLSIPSAIHLTRTIFRRIKLNLAWACIYNIVGLPIAMGFFLPFGIHMHPMFAGFAMACSSISVVVSSLALRWWQRPQWMDEASEPAGGLRWMSGTGIVGWAKETFGRVRRGKREEGYVALENLEV

SEQ ID NO: 7 - DNA, 2404 bp, T. reesei
atgctgttgtcagcgtccctctcggcgttggccttggccacagtttcactcgcacagggcacgacacacatccccgtcaccggtgttcccgtctctcctggtgctgccgtgccgctgagacagaacatcaatgacctggccaagtccgggccgcaatggtgagtgacgccctccttccaccacactttacctcagtcaagagacaagagggagacaagtacaaagcggatgaaaagaggtggacaagagagagagagagagaaagtgtgtgtgtgtatgtgagagcgagagagagagagagagacaagagctattggatggaccaggagccagcatggagaacagggggagacttgacgattcgaggagaggggggctcacatgtgcgtgcgaatagggatctctacgttcaggccatgtacaacatgtccaagatggactcccatgacccgtacagcttcttccagattgccggtaaatatacatctcggcctcctgcgaggcgacgtgactctcggagcttttagtaacaccagctaggcatccacggcgcaccgtacattgagtacaacaaggccggagcaaagtcgggcgatggctggctgggctactgccctcacggtgtatgtgtttttgtccatcgaggagggcgcaagagtttcatggacttgaactcttcgcccttgttgtgagccggaaatcatcgtctctgacagtttcattaggaggacctcttcatcagctggcaccgcccctatgtcctgctctttgaggtatgatttgaccacgctggactttgacctcatacaaacatcaactgacatcgttgcagcaagccttggtctccgtcgccaagggcatcgccaactcgtatcccccgtctgtccgcgccaagtaccaggctgccgccgccagcctgcgcgccccctactgggactgggccgccgacagctccgtgcccgccgtcaccgtcccccagacgctcaagatcaacgtccccagcggcagcagcaccaagaccgtcgactacaccaacccgctcaagacgtactacttcccgcgcatgtccttgaccggctcgtacggcgagttcaccggcggaggcaacgaccacaccgtccgctgcgccgcctccaagcagagctatcccgccaccgccaactccaacctggctgcccgtccttacaagtcctggatcgtacgtagtccccctttccctttggaagcttccccttgagtaaagctcgtcactgacacagagagcggcccgcagtacgatgtcctgaccaactctcaaaactttgccgacttcgcctccaccagcggccccggcatcaacgttgagcagatccacaacgccatccactgggacggtgcttgcggctcccagttcctcgcccccgactactccggcttcgaccccctgttgtaagtcaatcgagacgtcaagagtcatcttgtcaacaaccgatggcaaacgcagtctgtactgacgctgcaaaatagcttcatgcaccacgcccaggtcgaccgcatgtgggccttctgggaggccatcatgccctcgtcgcccctcttcacggcctcgtacaagggccagtcgcgcttcaactccaagtcgggcagcaccatcacccccgactcgcccctgcagcccttctaccaggccaacggcaagttccacacgtccaacacggtcaagagcatccagggcatgggctactcgtaccagggcatcgagtactggcaaaagtcccaggcccagatcaagtcgagcgtcaccaccatcatcaaccagctgtacgggcccaactcgggcaagaagcgcaacgccccgcgcgacttcttgagcgacattgtcaccgacgtcgagaacctcatcaagacccgttactttgccaagatctcggtcaacgtgaccgaggtgacggtccgccccgccgagatcaacgtctacgtcggcggccagaaggccggcagcttgatcgtcatgaagctccccgccgagggcacggtcaacggcggcttcaccattgacaaccccatgcaaagcatcctgcacggtggtctccgcaacgccgtccaggcctttaccgaggacattgaggttgagattctctctgtaagttttcccccctctctccactcccgaccactcactgtcactatttcgactagtcaccgtcaagatgtgtatttgtttgctgacccccaagcgcagaaggacggacaagccatccccctcgagacggtccccagcctgtccatcgacctcgaggtcgccaacgtcaccctgccctccgccctcgaccagctgcccaagtacggccagcgctccaggcaccgcgccaaggccgcccagcgcggacaccgctttgccgttccccatatccctcctctgtaa

SEQ ID NO: 8 - DNA, 1686 bp, T. reesei
atgctgttgtcagcgtccctctcggcgttggccttggccacagtttcactcgcacagggcacgacacacatccccgtcaccggtgttcccgtctctcctggtgctgccgtgccgctgagacagaacatcaatgacctggccaagtccgggccgcaatgggatctctacgttcaggccatgtacaacatgtccaagatggactcccatgacccgtacagcttcttccagattgccggcatccacggcgcaccgtacattgagtacaacaaggccggagcaaagtcgggcgatggctggctgggctactgccctcacggtgaggacctcttcatcagctggcaccgcccctatgtcctgctctttgagcaagccttggtctccgtcgccaagggcatcgccaactcgtatcccccgtctgtccgcgccaagtaccaggctgccgccgccagcctgcgcgccccctactgggactgggccgccgacagctccgtgcccgccgtcaccgtcccccagacgctcaagatcaacgtccccagcggcagcagcaccaagaccgtcgactacaccaacccgctcaagacgtactacttcccgcgcatgtccttgaccggctcgtacggcgagttcaccggcggaggcaacgaccacaccgtccgctgcgccgcctccaagcagagctatcccgccaccgccaactccaacctggctgcccgtccttacaagtcctggatctacgatgtcctgaccaactctcaaaactttgccgacttcgcctccaccagcggccccggcatcaacgttgagcagatccacaacgccatccactgggacggtgcttgcggctcccagttcctcgcccccgactactccggcttcgaccccctgttcttcatgcaccacgcccaggtcgaccgcatgtgggccttctgggaggccatcatgccctcgtcgcccctcttcacggcctcgtacaagggccagtcgcgcttcaactccaagtcgggcagcaccatcacccccgactcgcccctgcagcccttctaccaggccaacggcaagttccacacgtccaacacggtcaagagcatccagggcatgggctactcgtaccagggcatcgagtactggcaaaagtcccaggcccagatcaagtcgagcgtcaccaccatcatcaaccagctgtacgggcccaactcgggcaagaagcgcaacgccccgcgcgacttcttgagcgacattgtcaccgacgtcgagaacctcatcaagacccgttactttgccaagatctcggtcaacgtgaccgaggtgacggtccgccccgccgagatcaacgtctacgtcggcggccagaaggccggcagcttgatcgtcatgaagctccccgccgagggcacggtcaacggcggcttcaccattgacaaccccatgcaaagcatcctgcacggtggtctccgcaacgccgtccaggcctttaccgaggacattgaggttgagattctctctaaggacggacaagccatccccctcgagacggtccccagcctgtccatcgacctcgaggtcgccaacgtcaccctgccctccgccctcgaccagctgcccaagtacggccagcgctccaggcaccgcgccaaggccgcccagcgcggacaccgctttgccgttccccatatccctcctctgtaa

SEQ ID NO: 9 - protein, 561 aa, T. reesei
MLLSASLSALALATVSLAQGTTHIPVTGVPVSPGAAVPLRQNINDLAKSGPQWDLYVQAMYNMSKMDSHDPYSFFQIAGIHGAPYIEYNKAGAKSGDGWLGYCPHGEDLFISWHRPYVLLFEQALVSVAKGIANSYPPSVRAKYQAAAASLRAPYWDWAADSSVPAVTVPQTLKINVPSGSSTKTVDYTNPLKTYYFPRMSLTGSYGEFTGGGNDHTVRCAASKQSYPATANSNLAARPYKSWIYDVLTNSQNFADFASTSGPGINVEQIHNAIHWDGACGSQFLAPDYSGFDPLFFMHHAQVDRMWAFWEAIMPSSPLFTASYKGQSRFNSKSGSTITPDSPLQPFYQANGKFHTSNTVKSIQGMGYSYQGIEYWQKSQAQIKSSVTTIINQLYGPNSGKKRNAPRDFLSDIVTDVENLIKTRYFAKISVNVTEVTVRPAEINVYVGGQKAGSLIVMKLPAEGTVNGGFTIDNPMQSILHGGLRNAVQAFTEDIEVEILSKDGQAIPLETVPSLSIDLEVANVTLPSALDQLPKYGQRSRHRAKAAQRGHRFAVPHIPPL

SEQ ID NO: 10 - protein, 505 aa, Cerrena unicolor
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 11 - protein, 323 aa, T. reesei
HGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 12 - protein, 178 aa, T. reesei
MDMGDGSSQSCKISMLWNWYTVDACFLSSSWRIRNRGMFAASCIGIVLLVASVELMRRIGQEYDNSIVRQWHRQAAMASDRAGGRTQGSASYCERLLFRATPLQQLVRAIIHAATFGAAYIVMLLAMYFNGYIIICIIVGSGVGKFACHWLSVEIDLQPGEGERLLPKPILQTTICCD

SEQ ID NO: 13 - protein, 124 aa, T. reesei
MLWNWNVMNTCFISKHWQITSKGMFAGSCIGVILLVIALEFLRRLSKEYDRFLIKQHAAPRAVPAFRPSVLQQALRALLHVAQFSVAYIVMLLAMYYNGYFIICIFIGAYIGSFVFHWEPLTAG

SEQ ID NO: 14 - protein, 159 aa, T. reesei
MDHSHHMHAMEGHEGHGGHGGGMQDMCSMNMLFTWDTTNLCIVFRQWHVRSTASLIFSLIAVVLLGIGYEALRSVSRRYEASLATRLETVPRQNRETVSKRGHVIKATLYAIQNFYAFMLMLVFMTYNGWVMVAVSLGAFVGYLLFGHSTSATKDNACH

SEQ ID NO: 15 - protein, 172 aa, T. reesei
MTMLMAMVFQTDIRTPLYANSWTPHHAGAYAGTCIFLIALAVIARLLVAFRARQERIWADHDARRRYVVVNGKEPVAERLSRDSDAKSATMVISENGVEERVVVVEKKDGATRPWRFSVDPVRAAMDTVIVGVGYLLMLAVMTMNVGYFMSVLGGTFLGSLLVGRYSEVYHH

(19) United States
(12) Patent Application Publication
(10) Pub. No.: US 2017/0233746 A1
(43) Pub. Date: Aug. 17, 2017

(54) COMPOSITIONS AND METHODS FOR IMPROVED PROTEIN PRODUCTION

(71) Applicant: DANISCO US INC., Palo Alto, CA (US)

(72) Inventor: Susan M. MADRID, Millbrae, CA (US)

(73) Assignee: DANISCO US INC., Palo Alto, CA (US)

(21) Appl. No.: 15/503,859

(22) PCT Filed: Aug. 14, 2015
(86) PCT No.: PCT/US2015/045260

§ 371 Date: Feb. 14, 2017

Related U.S. Application Data
(60) Provisional application No. 62/038,095, filed on Aug. 15, 2014.

(51) Int. Cl. C12N 15/52; C12N 9/02; C12N 9/06; C12N 9/04

(52) U.S. Cl. CPC C12N 15/52; C12Y 101/99018; C12Y 110/03002; C12Y 114/18001; C12Y 104/03002; C12Y 103/03005; C12Y 110/03001; C12Y 101/03009; C12Y 101/03005; C12Y 110/03003; C12Y 114/17001; C12Y 114/17003; C12Y 113/11024; C12Y 115/01001; C12N 9/0006; C12N 9/0061; C12N 9/0071; C12N 9/0022; C12N 9/001; C12N 9/0059; C12N 9/0063; C12N 9/0069; C12N 9/0089

(57) ABSTRACT

Aspects of the present disclosure are drawn to methods of improving the expression of secreted cuproenzymes from host cells by manipulating the expression level of one or more proteins involved in copper transport in the host cell, e.g., membrane-bound copper transporting ATPases and soluble copper transporters. The present disclosure also provides compositions containing such improved host cells as well as products derived from the improved host cells that contain one or more cuproenzymes of interest.

### CROSS-REFERENCE TO RELATED APPLICATIONS

The present application claims priority to U.S. Provisional Patent Appln. Ser. No. 62/038,095, filed Aug. 15, 2014, which is incorporated herein by reference in its entirety.

### SEQUENCE LISTING

The sequence listing submitted via EFS, in compliance with 37 C.F.R. §1.52(e), is incorporated herein by reference. The sequence listing text file submitted via EFS contains the file “40456-WO-PCT_ST25.txt” created on Jul. 10, 2015, which is 44 kilobytes in size.

### FIELD OF THE INVENTION

Aspects of the present disclosure are drawn to methods of improving the expression of secreted cuproenzymes from host cells by manipulating the expression level of one or more copper metallochaperones, e.g., membrane-bound copper transporting ATPases and soluble copper transporters. The present disclosure also provides compositions containing such improved host cells as well as products made from the improved host cells that contain one or more cuproenzyme(s) of interest.

### INTRODUCTION

Copper is a redox active transition metal that is an essential co-factor for numerous enzymes (referred to herein as cuproenzymes). However, the level of free copper in a cell must be kept at low levels due to its toxicity. As such, less than 0.01% of the total cellular copper is free in the cytoplasm; most copper is bound and chelated by metallothioneins to prevent its cell-toxic effects. In addition, different compartments in the cell have different levels of copper, with the mitochondria having greater levels of copper than the cytoplasm, which in turn has greater levels than the Golgi apparatus.

The limited availability of free copper in cells is problematic in industrial settings for producing one or more functional cuproenzymes in recombinant host cells that have been engineered to over-express such enzymes. Due to the cellular copper gradient noted above, this issue is particularly evident when producing secreted cuproenzymes. However, it is a considerable technical challenge to provide additional copper during host cell culture in amounts that strike the correct balance: promoting the production of functional and secreted cuproenzymes without becoming toxic to the host cells.

In addition to the issues related to the production of cuproenzymes from host cells, the level of copper permitted in waste water discharged from industrial plants is regulated. As such, there is also an upper limit to how much copper can be added to a cuproenzyme fermentation process.

There is thus a need to develop recombinant host cells and methods of using such host cells to improve the production of cuproenzymes in fermentation processes.

### SUMMARY

Aspects of the present invention are based, at least in part, on the discovery that increased expression of one or more copper metallochaperones in a desired recombinant host cell, e.g., a filamentous fungal host cell, can improve secreted cuproenzyme production in a host cell. Accordingly, provided herein are recombinant host cells with increased expression of one or more copper metallochaperones that exhibit improved cuproenzyme production/secretion as compared to a parent host cell that does not have increased expression of the one or more copper metallochaperones, under substantially the same culture conditions. Methods of producing cuproenzymes from these host cells as well as compositions containing cuproenzymes produced from such host cells are also provided. Examples of secreted cuproenzymes that find use in the subject compositions and methods include, without limitation, lytic polysaccharide mono-oxygenases (LPMO), laccases, tyrosinases, amine oxidases, bilirubin oxidases, catechol oxidases, dopamine beta-monooxygenases, galactose oxidases, hexose oxidases, L-ascorbate oxidases, peptidylglycine monooxygenases, polyphenol oxidases, quercetin 2,3-dioxygenases, and superoxide dismutases.

Aspects of the present invention include, but are not limited to, the following:

1. A method for producing a cuproenzyme from a host cell comprising: overexpressing a copper metallochaperone in a host cell that expresses a cuproenzyme, and culturing the host cell under conditions sufficient to produce the cuproenzyme, wherein the host cell produces an increased amount of the cuproenzyme as compared to a corresponding host cell that does not overexpress the copper metallochaperone when cultured under substantially the same culture conditions.

2. The method of 1, wherein the cuproenzyme is secreted from the host cell.

3. The method of 1 or 2, wherein the cuproenzyme is selected from the group consisting of: a lytic polysaccharide mono-oxygenase (LPMO), a laccase, a tyrosinase, an amine oxidase, a bilirubin oxidase, a catechol oxidase, a dopamine beta-monooxygenase, a galactose oxidase, a hexose oxidase, a L-ascorbate oxidase, a peptidylglycine monooxygenase, a polyphenol oxidase, a quercetin 2,3-dioxygenase, and a superoxide dismutase.

4. The method of any above, wherein the cuproenzyme is endogenous to the host cell.

5. The method of any above, wherein the cuproenzyme is heterologous to the host cell.

6. The method of any above, wherein expression of the cuproenzyme and/or the copper metallochaperone is controlled by a promoter derived from the host cell.

7. The method of 6, wherein the host cell is a Trichoderma reesei (T. reesei) cell and the promoter is a pyruvate kinase (pki) or cellobiohydrolase I (cbh1) promoter derived from T. reesei.

8. The method of any above, wherein the host cell expresses at least one additional cuproenzyme, wherein the production of the at least one additional cuproenzyme is increased as compared to a corresponding host cell that does not overexpress the copper metallochaperone under substantially the same culture conditions.

9. The method of any above, wherein the copper metallochaperone is a membrane-bound copper transporting ATPase.

10. The method of 9, wherein the membrane-bound copper transporting ATPase comprises an amino acid sequence that is at least 60% identical to SEQ ID NO:6.

11. The method of 9 or 10, wherein the membrane-bound copper transporting ATPase is selected from Table 2.

12. The method of any one of 1-8, wherein the copper metallochaperone is a soluble copper transporter.

13. The method of 12, wherein the soluble copper transporter comprises an amino acid sequence that is at least 60% identical to SEQ ID NO:3.

14. The method of 12 or 13, wherein the soluble copper transporter is selected from Table 1.

15. The method of any above, further comprising over-expressing a second copper metallochaperone in the host cell.

16. The method of 15, wherein the first copper metallochaperone is a membrane-bound copper transporting ATPase comprising an amino acid sequence that is at least 60% identical to SEQ ID NO:6 and the second copper metallochaperone is a soluble copper transporter comprising an amino acid sequence that is at least 60% identical to SEQ ID NO:3.

17. The method of any above, wherein the host cell is a filamentous fungal host cell.

18. The method of 17, wherein the filamentous fungal host is selected from the group consisting of: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

19. The method of 17, wherein the filamentous fungal host cell is a Trichoderma reesei, an Aspergillus niger, an Aspergillus oryzae, or a Talaromyces emersonii host cell.

20. The method of any above, wherein the over-expressing step comprises increasing the expression of transcription factor Mac1 in the host cell.

21. The method of 20, wherein increasing the expression of Mac1 comprises introducing a Mac1 expression vector into the host cell.

22. A method of decreasing copper toxicity of a host cell comprising: over-expressing a copper metallochaperone in a host cell, wherein the host cell has decreased copper toxicity as compared to a corresponding host cell that does not overexpress the copper metallochaperone.

23. The method of 22, wherein the host cell over-expresses a cuproenzyme.

24. A method of reducing copper levels in a cell culture broth comprising: culturing a host cell over-expressing a copper metallochaperone in a cell culture media comprising copper to produce a cell culture broth, wherein the resulting level of copper in the cell culture broth is reduced as compared to a cell culture broth derived from a corresponding host cell that does not over-express the copper metallochaperone, in substantially the same cell culture media and cultured under substantially the same conditions.

25. A recombinant host cell comprising: a first polynucleotide encoding a cuproenzyme, and a second polynucleotide encoding a copper metallochaperone, wherein the cuproenzyme is expressed in the host cell and the copper metallochaperone is over-expressed in the host cell, and wherein the level of expression of the cuproenzyme is increased in the host cell as compared to a corresponding host cell that does not overexpress the copper metallochaperone under substantially the same culture conditions.

26. The recombinant host cell of 25, wherein the cuproenzyme is secreted from the host cell.

27. The recombinant host cell of 25, wherein the cuproenzyme is selected from the group consisting of: lytic polysaccharide monooxygenase (LPMO), a laccase, a tyrosinase, an amine oxidase, a bilirubin oxidase, a catechol oxidase, a dopamine beta-monooxygenase, a galactose oxidase, a hexose oxidase, a L-ascorbate oxidase, a peptidylglycine monooxygenase, a polyphenol oxidase, a quercetin 2,3-dioxygenase, and a superoxide dismutase.

28. The recombinant host cell of 27, wherein the cuproenzyme is selected from those listed in Table 3.

29. The recombinant host cell of any one of 25 to 28, wherein the cuproenzyme is heterologous to the host cell.

30. The recombinant host cell of any one of 25 to 29, wherein expression of the cuproenzyme and/or the copper metallochaperone is controlled by a promoter of the host cell.

31. The recombinant host cell of 30, wherein host cell is T. reesei and the promoter is a pki or a cbh1 promoter derived from T. reesei.

32. The recombinant host cell of any one of 25 to 31, wherein the second polynucleotide encodes a membrane-bound copper transporting ATPase comprising an amino acid sequence that is at least 60% identical to SEQ ID NO:6.

33. The recombinant host cell of any one of 25 to 32, wherein the second polynucleotide encodes a soluble copper transporter comprising an amino acid sequence that is at least 60% identical to SEQ ID NO:3.

34. The recombinant host cell of any one of 25 to 33, wherein the host cell further comprises a third polynucleotide encoding a second copper metallochaperone.

35. The recombinant host cell of 34, wherein the first copper metallochaperone is a membrane-bound copper transporting ATPase comprising an amino acid sequence that is at least 60% identical to SEQ ID NO:6 and the second copper metallopchaperone is a soluble copper transporter comprising an amino acid sequence that is at least 60% identical to SEQ ID NO:3.

36. The recombinant host cell of any one of 25 to 35, wherein the recombinant host cell is a filamentous fungal host cell.

37. The recombinant host cell of 36, wherein the filamentous fungal host is selected from the group consisting of: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

38. The recombinant host cell of 36, wherein the filamentous fungal host cell is a T. reesei, an A. niger, an A. oryzae, or a T. emersonii host cell.

39. The recombinant host cell of any of 25-38, wherein the recombinant host cell over-expresses Mac1, wherein the over-expression of Mac1 leads to the over-expression of the copper metallochaperone in the host cell.

40. A supernatant obtained from a culture of the recombinant host cell of one of 25 to 39.

41. A culture supernatant obtained using the method of any one of 1 to 21.

### BRIEF DESCRIPTION OF THE DRAWINGS

The skilled artisan will understand that the drawings are for illustration purposes only. The drawings are not intended to limit the scope of the present teaching in any way.

FIGS. 1A-1C. Schematics of the expression constructs for the copper metallochaperones derived from T. reesei. (FIG. 1A) Expression construct for the membrane-bound copper transporter ATPase. (FIG. 1B) Expression construct for the cytoplasmic (soluble) copper transporter. These copper metallochaperone genes were expressed using the constitutive pyruvate kinase (pki) promoter and included a terminator derived from the CBH1 gene. Selective marker (hphR) hygromycin resistance gene was used for selection of transformants harbouring the above plasmids. AmpR is the ampicillin resistance gene used in propagation of the plasmids in bacterial cells. (FIG. 1C) Expression vector for over-expressing T. reesei tyrosinase (amino acid sequence: SEQ ID NO:9). Tyrosinase was transcribed from the cbh1 promoter and was followed by a cbh1 transcriptional terminator.

FIG. 2. Analysis of extracellular protein expression in 14 liter scale fermentation of a tyrosinase-overproducing strain by SDS-PAGE. Cultivation time is shown at the bottom in hours and the beginning of the copper feed is indicated with an upward arrow. Tyrosinase and endoglucanase 6 protein bands are indicated at the left (Tyr and EG6, respectively). The copper-containing tyrosinase enzyme showed a peak production within 69 hours and decreased accumulation during the remaining time course. In contrast, the non-copper containing enzyme endoglucanase 6 (EG6) showed increasing accumulation over the entire time course.

FIG. 3. Effect of increasing levels of copper on tyrosinase expression. SDS-PAGE showing expression of tyrosinase (Tyr) in the presence of increasing amounts of copper (shown at the bottom of each lane). As seen in this figure, increasing the amount of copper sulphate to the growth media resulted in decreased synthesis of tyrosinase.

FIG. 4. Analysis of two different strains (Strains A and C, top panel and bottom panel, respectively) overproducing tyrosinase cultivated at different copper concentrations ranging from 0 to 1000 μM. The highest concentration of copper without adverse effect to protein production was approximately 151.1M. Copper levels above 15 μM lead to reduced tyrosinase production levels. Tyrosinase activity present in the culture supernatant was measured using tyrosine as substrate and detecting the formation of product at 286 nm (open bars) and 470 nm (filled bars).

FIG. 5. A spot assay for tyrosinase activity was used to detect tyrosinase activity present in these strains cultivated in the presence of high levels of copper (6 mM) in which no detectable tyrosinase was produced. Tyrosinase activity could not be detected in the control wells for Strains A (wells in lane 8) and C (wells in lane 1), outlined with dotted lines. The ability of Strains A and C to produce tyrosinase was restored when these strains were retransformed with either the membrane-bound copper transporting ATPase expressing plasmid (wells in lanes 2-7) or the cytoplasmic (soluble) copper transporter expressing plasmid (wells in lanes 9-12). Thus, expression of either of these copper metallochaperone can reduce copper toxicity and resulted in expression of the tyrosinase cuproenzyme. Tyrosinase activity was detected in this assay by combining 10 μL of culture supernatant and 200 μL of 10% skim milk (pre-heated to 35° C.) in a microtiter plate and incubating the mixture for at least 10 minutes at 35° C. The milk turned from white to red when tyrosinase was present and active. Plus signs indicate wells with detectable red color.

FIG. 6. Expression vector construct for copper metalloprotein laccase D from Cerrena unicolor showing the laccase D gene transcribed from the cbh1 promoter with a CBH1 signal sequence and cbh1 transcriptional terminator. The mature laccase D sequence is SEQ ID NO: 10.

FIGS. 7A-7C. Analysis of laccase D production in a strain overexpressing laccase D (Strain 32A) both with and without over-expression of copper metallochaperones. FIG. 7A shows relative expression levels of laccase D in Strain 32A (leftmost bar; set at 100%) and strains (#46, #47, and #48) derived therefrom which overexpress both cytosolic transporter and membrane-bound copper transporting ATPase (transformed with the expression vectors shown in FIGS. 1A and 1B). FIG. 7B shows relative expression levels of laccase D in Strain 32A (leftmost bar; set at 100%) and strains (#2, #16, #29, #30 and #31) derived therefrom which overexpress the membrane-bound copper transporting ATPase (transformed with the expression vector shown in FIG. 1A). FIG. 7C shows relative expression levels of laccase D in Strain 32A (leftmost bar; set at 100%) and strains (#5, #22, #27 and #35) derived therefrom which overexpress the cytosolic copper transporter (transformed with the expression vector shown in FIG. 1B).

### DETAILED DESCRIPTION

Copper metallochaperones, both cytoplasmic (soluble) and membrane bound, function to bind to and transport copper to intracellular locations where it can be incorporated into copper metallo-proteins (e.g., cuproenzymes) (see, e.g., O'Halloran et al., Metallochaperones, an intracellular shuttle service, for metal ions. 2000 JBC: 275 (33):25057-25060; and Robinson et al., Copper Metallochaperones 2010 Annu. Rev. Biochem. 79:537-62). For secreted cuproenzymes, the action of multiple copper metallochaperones transport copper to the lumen of the Golgi complex, including cytosolic copper transporter (e.g., the yeast Atx1 polypeptide and homologs thereof) and Golgi membrane-bound copper permeases (e.g., the yeast Ccc2 polypeptide and homologs thereof). In the Golgi, the copper can be incorporated into cuproenzymes during the expression/folding/secretion process. (See, e.g., Huffman et al. Energetics of Copper Trafficking between Atx1 metallochaperone & the intracellular Copper transporter, Ccc2. 2000 JBC 275(25). 18611-18614.) Copper metallochaperones are highly conserved between all eukaryotes analysed.

The present teachings are based on the discovery that cuproenzyme secretion in a host cell can be improved by overexpressing one or more copper metallochaperones. Accordingly the present teachings provide methods for increasing protein secretion in a host cell, e.g., filamentous fungi, by overexpressing one or more copper metallochaperones, e.g., either a soluble copper transporter, a membrane bound copper transporter, or both. The present teachings also provide expression hosts, e.g., filamentous fungi containing certain copper metallochaperone(s) and a cuproenzyme of interest for increased secretion.

Before the present compositions and methods are described in greater detail, it is to be understood that the present compositions and methods are not limited to particular embodiments described, and as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior invention. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

### Definitions

The term “coding sequence” is defined herein as a nucleic acid sequence that, when placed under the control of appropriate control sequences including a promoter, is transcribed into mRNA which can be translated into a polypeptide. A coding sequence may contain a single open reading frame, or several open reading frames separated by introns, for example. A coding sequence may be cDNA, genomic DNA, synthetic DNA or recombinant DNA, for example. A coding DNA sequence generally starts at a start codon (e.g., ATG) and ends at a stop codon (e.g., TAA, TAG and TGA).

A “copper metallochaperone” or “copper chaperone” as used herein is a protein that facilitates the transport and/or the incorporation of copper into copper-requiring metallo-enzymes (also called cuproenzymes) in a cell. Copper metallochaperones include cytosolic (or soluble) copper transporters (e.g., SEQ ID NO:3 and Table 1), membrane-bound copper transporters (e.g., SEQ ID NOs: 12, 13, 14, and 15; homologs thereof; and sequences having at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identity thereto that retain copper transport activity), membrane bound transporting ATPase (e.g., SEQ ID NO:6 and Table 2). The latter includes copper metallochaperones that are present in the Golgi membrane which transport copper to proteins that are to be secreted from the host cell (and are also referred to as “copper permeases”, “copper transporter ATPases”, and the like).

A “cuproenzyme” is any metalloenzyme that contains one or more copper atoms. Examples include, but are not limited to, lytic polysaccharide mono-oxygenases (LPMO), laccases, tyrosinases, amine oxidases, bilirubin oxidases, catechol oxidases, dopamine beta-monooxygenases, galactose oxidases, hexose oxidases, L-ascorbate oxidases, peptidylglycine monooxygenases, polyphenol oxidases, quercetin 2,3-dioxygenases, and superoxide dismutases.

The term “derived from” encompasses the terms “originated from,” “obtained from,” “obtainable from,” “isolated from,” and “created from,” and generally indicates that one specified material find its origin in another specified material or has features that can be described with reference to another specified material.

The term “DNA construct” as used herein means a polynucleotide that comprises at least two adjoined DNA polynucleotide fragments.

The term “endogenous” with reference to a polynucleotide or polypeptide refers to a polynucleotide or polypeptide that occurs naturally in the host cell.

The term “expression” refers to the process by which a polypeptide is produced based on a nucleic acid sequence. The process includes both transcription and translation.

As used herein, “expression vector” means a DNA construct including a DNA sequence that encodes one or more specified polypeptides that are operably linked to a suitable control sequence capable of affecting the expression of the one or more polypeptides in a suitable host. Such control sequences may include a promoter to affect transcription, an optional operator sequence to control transcription, a sequence encoding suitable ribosome-binding sites on the mRNA, and sequences which control termination of transcription and translation. Different cell types may be used with different expression vectors. An exemplary promoter for vectors used in Bacillus subtilis is the AprE promoter; an exemplary promoter used in Streptomyces lividans is the A4 promoter (from Aspergillus niger); an exemplary promoter used in E. coli is the Lac promoter, an exemplary promoter used in Saccharomyces cerevisiae is PGK1, an exemplary promoter used in Aspergillus niger is glaA, and exemplary promoters for T. reesei include pki and cbhI. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the present compositions and methods are intended to include other forms of expression vectors which serve equivalent functions and which are, or become, known in the art. Thus, a wide variety of host/expression vector combinations may be employed in expressing the DNA sequences described herein.

Useful expression vectors, for example, may consist of segments of chromosomal, non-chromosomal and synthetic DNA sequences such as various known derivatives of SV40 and known bacterial plasmids, e.g., plasmids from E. coli including col E1, pCR1, pBR322, pMb9, pUC 19 and their derivatives, wider host range plasmids, e.g., RP4, phage DNAs e.g., the numerous derivatives of phage X, e.g., NM989, and other DNA phages, e.g., M13 and filamentous single stranded DNA phages, yeast plasmids such as the 2μ plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present compositions and methods are known in the art and are described generally in, for example, Sambrook et al., Molecular Cloning: A Laboratory Manual, Second Edition, Cold Spring Harbor Press (1989). Often, such expression vectors including the DNA sequences described herein are transformed into a unicellular host by direct insertion into the genome of a particular species through an integration event (see e.g., Bennett & Lasure, More Gene Manipulations in Fungi, Academic Press, San Diego, pp. 70-76 (1991) and articles cited therein describing targeted genomic insertion in fungal hosts).

The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, glucans, and other complex polysaccharides. The filamentous fungi of the present teachings are morphologically, physiologically, and genetically distinct from yeasts. Vegetative growth by filamentous fungi is by hyphal elongation and carbon catabolism is obligatory aerobic. Filamentous fungi include all filamentous forms of the subdivision Eumycotina, particulary Pezizomycotina species. A filamentous fungal parent cell may be a cell of a species of, but not limited to, Trichoderma, e.g., Trichoderma longibrachiatum, Trichoderma viride, Trichoderma koningii, Trichoderma harzianum; Penicillium sp.; Humicola sp., including Humicola insolens and Humicola grisea; Chrysosporium sp., including C. lucknowense; Myceliophthora sp.; Gliocladium sp.; Aspergillus sp.; Fusarium sp., Neurospora sp., Hypocrea sp., e.g., Hypocrea jecorina, and Emericella sp. As used herein, the term “Trichoderma” or “Trichoderma sp.” refers to any fungal strains which have previously been classified as Trichoderma or are currently classified as Trichoderma. In certain embodiments, a GH61 enzyme can be from a non-filamentous fungal cell. Examples of GH61A enzymes include those found in Hypocrea jecorina (Trichoderma reesei), Hypocrea rufa, Hypocrea orientalis, Hypocrea atroviridis, Hypocrea virens, Emericella nidulans, Aspergillus terreus, Aspergillus oryzae, Aspergillus niger, Aspergillus kawachii, Aspergillus flavus, Aspergillus clavatus, Gaeumannomyces graminis, Trichoderma saturnisporum, Neurospora tetrasperma, Neurospora crassa, Neosartorya fumigate, Neosartorya fumigate, Neosartorya fischeri, Thielavia terrestris, and Thielavia heterothallica.

The term “heterologous” refers to elements that are not normally associated with each other. For example, if a recombinant host cell produces a heterologous protein, that protein is not produced in a wild-type host cell of the same type, a heterologous promoter is a promoter that is not present in nucleic acid that is endogenous to a wild type host cell, and a promoter operably linked to a heterologous coding sequence is a promoter that is operably linked to a coding sequence that it is not usually operably linked to in a wild-type host cell.

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

By “homolog” or “homologous” is meant biomolecule has a specified degree of identity with the subject amino acid sequence(s) or the subject nucleotide sequence(s) indicated. A homologous sequence is taken to include an amino acid or nucleic acid sequence that is at least 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologs of a subject enzyme will include the same/similar active site residues as the subject enzyme and/or exhibit similar enzymatic activity unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J. Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J. Mol. Biol. 215:403-410).

Computerized programs using these algorithms are also available, and include, but are not limited to: ALIGN or Megalign (DNASTAR) software, or WU-BLAST-2 (Altschul et al., Meth. Enzym., 266:460-480 (1996)); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared. Preferably, the sequence identity is determined using the default parameters determined by the program. Specifically, sequence identity can determined by using Clustal W (Thompson J. D. et al. (1994) Nucleic Acids Res. 22:4673-4680) with default parameters, i.e.:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

As used herein, “host cell” or “host strain” means a cell suitable for a particular purpose, e.g., for expressing a particular gene, for propagating a vector, etc. In certain embodiments, a host cell harbors an expression vector including a polynucleotide sequence that encodes one or more proteins of interest according to the present compositions and methods (e.g., a polynucleotide sequence encoding a cuproenzyme and/or one or more copper metallochaperones). Host cells include both prokaryotic and eukaryotic organisms, including any transformable microorganism that finds use in expressing a desired polypeptide/enzyme (or multiple polypeptides/enzymes) and/or for propagation of a vector. Examples of host cells include, but are not limited to, species of Bacillus, Streptomyces, Escherichia, Trichoderma, Aspergillus, Saccharomyces, etc. In certain aspects, host cells are recombinant host cells, i.e., cells that are not found in nature (see definition of “recombinant” below).

The term “introduced” in the context of inserting a nucleic acid sequence into a cell, means “transfection”, “transformation” or “transduction,” as known in the art.

As used herein, “percent (%) sequence identity” with respect to an amino acid or nucleotide sequence is defined as the percentage of amino acid residues or nucleotides in a candidate sequence that are identical with the amino acid residues or nucleotides in a sequence of interest (e.g., a metallochaperone protein sequence), after aligning the sequences and introducing gaps, if necessary, to achieve the maximum alignment (percent sequence identity), and not considering any conservative substitutions as part of the sequence identity.

By “purified” or “isolated” or “enriched” is meant that a biomolecule (e.g., a polypeptide or polynucleotide) is altered from its natural state by virtue of separating it from some or all of the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition (e.g., purified polypeptide) which provide additional benefits, for example, activating agents, anti-inhibition agents, desirable ions, compounds to control pH or other enzymes or chemicals.

As used herein, “microorganism” refers to a bacterium, a fungus, a virus, a protozoan, and other microbes or microscopic organisms.

The term “nucleic acid” and “polynucleotide” are used interchangeably and encompass DNA, RNA, cDNA, single stranded or double stranded and chemical modifications thereof. Because the genetic code is degenerate, more than one codon may be used to encode a particular amino acid, and the present invention encompasses all polynucleotides, which encode a particular amino acid sequence.

The term “operably linked” refers to an arrangement of elements that allows them to be functionally related. For example, a promoter is operably linked to a coding sequence if it controls the transcription of the sequence, and a signal sequence is operably linked to a protein if the signal sequence directs the protein through the secretion system of a host cell.

As used herein, the terms “polypeptide” and “enzyme” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “promoter” is defined herein as a nucleic acid that directs transcription of a downstream polynucleotide in a cell. In certain cases, the polynucleotide may contain a coding sequence and the promoter may direct the transcription of the coding sequence into translatable RNA.

The term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell (or host cell) encompasses a cell that expresses one or more genes that are not found in its native parent (i.e., non-recombinant) cell, a cell that expresses one or more native genes in an amount that is different than its native parent cell, and/or a cell that expresses one or more native genes under different conditions than its native parent cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

The term “signal sequence” refers to a sequence of amino acids at the N-terminal portion of a protein, which facilitates the secretion of the mature form of the protein outside the cell. The mature form of the extracellular protein lacks the signal sequence which is cleaved off during the secretion process.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, expression cassettes and the like. Expression vectors and cassettes may include regulatory sequences such as promoters, signal sequences, coding sequences and transcription terminators.

The phrase “substantially the same culture conditions” and the like means that the conditions under which a first host cell is cultured are the same or nearly the same as those used for a second host cell such that a meaningful comparison of the performance or characteristic of the first and second host cells may be made. Parameters that are to be substantially the same include temperature, pH, copper concentration, time, agitation, culture media, etc. Setting up comparative host cell cultures that are performed under “substantially the same culture conditions” is well within the abilities of a person having ordinary skill in the art.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

Laccases (IUBMB Enzyme Nomenclature: EC 1.10.3.2) are copper-containing oxidase enzymes that are found in many plants, fungi, and microorganisms. Laccases act on phenols and similar molecules, performing one-electron oxidations. Laccases may play a role in the formation of lignin by promoting the oxidative coupling of monolignols, a family of naturally occurring phenols. Laccase is also referred to as: urishiol oxidase; urushiol oxidase; and p-diphenol oxidase.

Tyrosinases (IUBMB Enzyme Nomenclature: EC 1.14.18.1) are type III copper protein found in a broad variety of bacteria, fungi, plants, insects, crustaceans, and mammals, and is involved in the synthesis of a number of pigment molecules, e.g., betalains and melanin. Tyrosinase is also referred to as: monophenol monooxygenase; phenolase; monophenol oxidase; cresolase; monophenolase; tyrosine-dopa oxidase; monophenol monooxidase; monophenol dihydroxyphenylalanine:oxygen oxidoreductase; N-acetyl-6-hydroxytryptophan oxidase; monophenol, dihydroxy-L-phenylalanine oxygen oxidoreductase; o-diphenol:O₂ oxidoreductase; and phenol oxidase.

By “GH61” or “GH61 enzyme” or “AA9” or “AA9 enzyme” and the like is meant an enzyme that belongs to the glycoside hydrolase 61 family (GH61) which has recently been re-classified as AA9. AA9 (formerly GH61) proteins are copper-dependent lytic polysaccharide monooxygenases (LPMOs). A description of the AA9 family as well as a list of AA9 enzymes can be found at the Carbohydrate-Active Enzyme Database (CAZy) at www.cazy.org (see also Lombard V, Golaconda Ramulu H, Drula E, Coutinho P M, Henrissat B (2014) The Carbohydrate-active enzymes database (CAZy) in 2013. Nucleic Acids Res 42:D490-D495. [PMID: 24270786]). In certain aspects, an AA9 enzyme is derived from Trichoderma reesei and comprises the amino acid sequence shown in SEQ ID NO: 11, an amino acid sequence having at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identity thereto, an allelic variant thereof, or a fragment thereof that retains LPMO activity. A list accession numbers (Genbank and Uniprot) for GH61/AA9 family members from different species are provided in Table 3.

### Compositions and Methods

The present teachings are based on the discovery that cuproenzyme secretion in a host cell can be improved by overexpressing one or more copper metallochaperones. Accordingly the present teachings provide methods for increasing protein secretion in a host cell, e.g., filamentous fungi, by overexpressing one or more copper metallochaperones, e.g., either a soluble copper transporter, a membrane bound copper transporter, or both. The present teachings also provide expression hosts, e.g., filamentous fungi containing certain copper metallochaperone(s) and a cuproenzyme of interest for increased secretion.

According to one aspect of the present teachings, methods are provided for increasing the secretion/production of a cuproenzyme of interest in a host by overexpressing a copper metallochaperone along with the desired cuproenzyme in the host cell. The copper metallochaperone of the present teachings can be any suitable protein associated with copper transport. In some embodiments, the copper metallochaperone can be a fragment of a copper metallochaperone with substantially the same, or enhanced, copper transporting function as the full-length copper metallochaperone.

In various embodiments, copper metallochaperones that find use in aspects of the present teachings include any cytosolic/soluble or membrane bound copper transporters. In some embodiments, the copper metallochaperone is selected from the copper transporters shown in Tables 1 and 2 and derivatives or homologs thereof, e.g., based on function or structure similarities commonly accepted by one skilled in the art. For example, certain aspects of the present invention include the use of one or more soluble copper transporters with an amino acid sequence identical or substantially identical, e.g., having at least 50%, 60%, 70%, 80%, 90%, 95%, 96%, 97%, 98%, 99% or greater % identity, to SEQ ID NO:3. In addition, certain aspects of the present invention include the use of one or more membrane bound copper transporters with an amino acid sequence identical or substantially identical, e.g., having at least 50%, 60%, 70%, 80%, 90%, 95%, 96%, 97%, 98%, 99% or greater % identity, to SEQ ID NO:6, 12, 13, 14 or 15. As detailed herein, host cells that exhibit improved cuproenzyme secretion can express one or more membrane bound copper transporters, one or more soluble copper transporters, or a combination of both membrane bound and soluble copper transporters.

In general, the one or more copper metallochaperones are overexpressed in a host cell along with one or more desired cuproenzymes in a host cell, where the expression of the copper metallochaperone and the cuproenzyme are under the control of their own respective operably-linked promoter. In some embodiments, the copper metallochaperone and/or cuproenzyme are expressed under a promoter native to the desired host cell or, alternatively, the copper metallochaperone and/or cuproenzyme are expressed under a promoter that is heterologous to the desired host cell. In some embodiments, the copper metallochaperone and/or cuproenzyme are expressed under a constitutive promoter whereas in other embodiments the copper metallochaperone and/or cuproenzyme are expressed under an inducible promoter. It is noted that any combination of promoters may be employed to express the copper metallochaperone (i.e., one or more copper metallochaperones) and the cuproenzyme (i.e., one or more cuproenzymes) in the host cell. For example, the one or more copper metallochaperones are expressed under a heterologous constitutive promoter whereas the one or more cuproenzymes are expressed under a native inducible promoter (or vice versa). In some embodiments, the operably-linked promoter can be a modified native promoter, e.g., mutated native promoter with enhanced transcription activity of the promoter.

In certain embodiments, overexpression of the one or more copper metallochaperones can be achieved by altering the expression of a transcriptional repressor or inducer of the native promoter of the one or more copper metallochaperones in a host cell. For example, the expression of a transcriptional repressor of a copper metallochaperone can be reduced in a host cell or, conversely, the expression of a transcriptional inducer (or activator) of a copper metallochaperone can be increased in a host cell. In but one example, the expression of the copper metallochaperone transcriptional activator Mac1 (Metal-binding activator 1; a copper deficiency-inducible transcription factor of yeast) can be increased in a host cell, thereby leading to overexpression of the copper metallochaperone. Increasing the expression of a transcriptional activator (e.g., Mac1) can be achieved by introducing an expression cassette or expression vector for the transcription factor into a host cell.

As used herein, the term “promoter” refers to a nucleic acid sequence that functions to direct transcription of an operably linked coding sequence (e.g., a gene, cDNA, or a synthetic coding sequence). A promoter can include necessary nucleic acid sequences near the start site of transcription, such as, in the case of a polymerase II type promoter, a TATA element. The promoter, together with other transcriptional and translational regulatory nucleic acid sequences, collectively referred to as regulatory sequences, controls the expression of the operably linked coding sequence. In general, the regulatory sequences include, but are not limited to, promoter sequences, ribosomal binding sites, transcriptional start and stop sequences, translational start and stop sequences, and enhancer or activator sequences. The regulatory sequences will generally be appropriate to and recognized by the host cell in which the coding sequence is being expressed.

A constitutive promoter is a promoter that is active under most environmental and developmental conditions. An inducible or repressible promoter is a promoter that is active under environmental or developmental regulation. Promoters can be inducible or repressible by changes in environment factors such as, but not limited to, carbon, nitrogen or other nutrient availability, temperature, pH, osmolarity, the presence of heavy metal, the concentration of an inhibitor, stress, or a combination of the foregoing, as is known in the art. Promoters can be inducible or repressible by metabolic factors, such as the level of certain carbon sources, the level of certain energy sources, the level of certain catabolites, or a combination of the foregoing, as is known in the art.

Suitable non-limiting examples of promoters include cbh1, cbh2, eg11, eg12, eg13, eg14, eg15, xyn1, and xyn2, repressible acid phosphatase gene (phoA) promoter of P. chrysogenum (see Graessle et al., Applied and Environmental Microbiology (1997), 63(2), 753-756), glucose-repressible PCK1 promoter (see Leuker et al. Gene (1997), 192(2), 235-240), maltose-inducible, glucose-repressible MRP1 promoter (see Munro et al. Molecular Microbiology (2001), 39(5), 1414-1426), methionine-repressible MET3 promoter (see Liu et al. Eukaryotic Cell (2006), 5(4), 638-649).

An example of an inducible promoter useful in the present teachings is the cbh1 promoter of Trichoderma reesei, the nucleotide sequence of which is deposited in GenBank under Accession Number D86235. Other exemplary promoters are promoters involved in the regulation of genes encoding cellulase enzymes, such as, but not limited to, cbh2, eg11, eg12, eg13, eg15, xyn1 and xyn2.

According to the present teachings, the copper metallochaperone can be used to increase the secretion/production of any suitable cuproenzyme in a host. The secretable cuproenzyme is generally operably linked to a signal sequence when first expressed in the host cell, e.g., an amino acid sequence tag leading proteins or polypeptides through the secretion pathway of a cell. The signal sequence can be the native signal sequence for the cuproenzyme (i.e., the signal sequence found in the wild-type enzyme) or a heterologous signal sequence (i.e., a signal sequence derived from a different secreted protein that is operably linked to the mature cuproenzyme of interest by recombinant methods). Any suitable signal sequence known or later discovered can be used, e.g., the signal sequences from A. niger glucoamylase or aspartic protease, or the signal sequence from Rhizomucor miehei or Trichoderma reesei aspartic proteases or cellulases, e.g., Trichoderma reesei cellobiohydrolase I, cellobiohydrolase II, endoglucanase I, endoglucanase II or endoglucanase III.

According to the present teachings, the copper metallochaperone can be used in any host to increase the secretion of a desired cuproenzyme in the host. In come embodiments, the expression hosts is a filamentous fungus. In general, a “filamentous fungus” is a eukaryotic microorganism that is the filamentous form of the subdivision Eumycotina. These fungi are characterized by a vegetative mycelium with a cell wall composed of chitin, beta-glucan, and other complex polysaccharides. In various embodiments, the filamentous fungi of the present teachings are morphologically, physiologically, and genetically distinct from yeasts. In some embodiments, the filamentous fungi of the present teachings include, but are not limited to the following genera: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus. In some embodiments, the filamentous fungi of the present teachings include, but are not limited to the following: A. nidulans, A. niger, A. awamori, A. oryzae, Hypocrea jecorina, N. crassa, Trichoderma reesei, and Trichoderma viride.

Another aspect of the present teachings provides an expression host expressing a copper metallochaperone and a desired cuproenzyme of interest. In some embodiments, the expression host of the present teachings contains a first polynucleotide encoding a cuproenzyme and a second polynucleotide encoding a copper metallochaperone. In some embodiments, the expression host further contains a third polynucleotide encoding a second copper metallochaperone, e.g., different from the one encoded by the second polynucleotide. In addition, the host cell can further include a fourth polynucleotide encoding a second cuproenzyme of interest, e.g., different from the one encoded by the first polynucleotide. In certain embodiments, the polynucleotides encoding the cuproenzyme(s) and the copper metallochaperone(s) are recombinant expression cassettes that have been introduced into the host cell, e.g., by transformation, and which are described in further detail below.

In some embodiments the desired cuproenzyme may be produced as a fusion polypeptide. In some embodiments the desired cuproenzyme may be fused to a polypeptide that is efficiently secreted by a filamentous fungus to enhance secretion, facilitate subsequent purification/identification or enhance stability.

In general, the one or more polynucleotides encoding the one or more copper metallochaperones and/or the one or more cuproenzymes in the expression host of the present teachings can be either genetically inserted or integrated into the genomic makeup of the expression host, e.g., integrated into the chromosome of the expression host, or existing extrachromosomally, e.g., existing as a replicating vector within the expression host under selection condition for a selection marker carried by the vector.

The production/secretion of a secretable cuproenzyme can be measured in a sample (e.g., a culture broth) directly, for example, by assays that detect for enzyme activity or the amount of the enzyme present. Immunological methods, such as Western blot or ELISA, can be used to qualitatively and quantitatively evaluate expression of a secretable cuproenzyme. The details of such methods are known to those of skill in the art and many reagents for practicing such methods are commercially available.

TABLE 1
List of proteins with homologies to the soluble (cytosolic) T. reesei copper
transporter (SEQ ID NO: 3). Table 1 shows the accession number (UNIPROT), organism and
sequence identity to SEQ ID NO: 3. The protein sequence database UNIPROT was used as
source of the amino acid sequences. Sequence identity was determined using a standard protein-
protein BLAST (blastp) against the Uniprot database on the NCBI/BLAST website.

| | | % ID to T. reesei |
|---|---|---|
| Accession | | Soluble |
| No. | | Copper |
| (UNIPROT) | Organism/Strain | Transporter |
| G0RSG6 | Hypocrea jecorina (strain QM6a) (Trichoderma reesei) | 100.00% |
| G9MGG2 | Hypocrea virens (strain Gv29-8/FGSC 10586) (Gliocladium | 88.00% |
| | virens) (Trichoderma virens) | |
| C7Z0W4 | Nectria haematococca (strain 77-13-4/ATCC MYA-4622/ | 88.00% |
| | FGSC 9596/MPVI) (Fusarium solani subsp. pisi) | |
| W9HYZ7 | Fusarium oxysporum FOSC 3-a | 83.00% |
| N4UNQ9 | Fusarium oxysporum f. sp. cubense (strain race 1) (Panama | 83.00% |
| | disease fungus) | |
| N1S578 | Fusarium oxysporum f. sp. cubense (strain race 4) (Panama | 83.00% |
| | disease fungus) | |
| J9NC66 | Fusarium oxysporum f. sp. lycopersici (strain 4287/CBS 123668/ | 83.00% |
| | FGSC 9935/NRRL 34936) (Fusarium vascular wilt of tomato) | |
| G3J9Z1 | Cordyceps militaris (strain CM01) (Caterpillar fungus) | 90.00% |
| E9ERN2 | Metarhizium anisopliae (strain ARSEF 23/ATCC MYA-3075) | 84.00% |
| S0EGT1 | Gibberella fujikuroi (strain CBS 195.34/IMI 58289/NRRL A- | 81.00% |
| | 6831) (Bakanae and foot rot disease fungus) (Fusarium fujikuroi) | |
| F9G5W7 | Fusarium oxysporum (strain Fo5176) (Fusarium vascular wilt) | 81.00% |
| J4UKW3 | Beauveria bassiana (strain ARSEF 2860) (White muscardine | 87.00% |
| | disease fungus) (Tritirachium shiotae) | |
| G9NWT7 | Hypocrea atroviridis (strain ATCC 20476/IMI 206040) | 81.00% |
| | (Trichoderma atroviride) | |
| F9XNY2 | Mycosphaerella graminicola (strain CBS 115943/IPO323) | 83.00% |
| | (Speckled leaf blotch fungus) (Septoria tritici) | |
| E9E111 | Metarhizium acridum (strain CQMa 102) | 84.00% |
| K3VY44 | Fusarium pseudograminearum (strain CS3096) (Wheat and barley | 75.00% |
| | crown-rot fungus) | |
| I1S268 | Gibberella zeae (strain PH-1/ATCC MYA-4620/FGSC 9075/ | 74.00% |
| | NRRL 31084) (Wheat head blight fungus) (Fusarium | |
| | graminearum) | |
| M1W946 | Claviceps purpurea (strain 20.1) (Ergot fungus) (Sphacelia | 81.00% |
| | segetum) | |
| T4ZYJ9 | Ophiocordyceps sinensis (strain Co18/CGMCC 3.14243) | 80.00% |
| | (Yarsagumba caterpillar fungus) (Hirsutella sinensis) | |
| T0KGZ7 | Colletotrichum gloeosporioides (strain Cg-14) (Anthracnose | 75.00% |
| | fungus) (Glomerella cingulata) | |
| L2G003 | Colletotrichum gloeosporioides (strain Nara gc5) (Anthracnose | 75.00% |
| | fungus) (Glomerella cingulata) | |
| E3QL83 | Colletotrichum graminicola (strain M1.001/M2/FGSC 10212) | 74.00% |
| | (Maize anthracnose fungus) (Glomerella graminicola) | |
| H1UVP4 | Colletotrichum higginsianum (strain IMI 349063) (Crucifer | 73.00% |
| | anthracnose fungus) | |
| N4VDA2 | Colletotrichum orbiculare (strain 104-T/ATCC 96160/CBS | 72.00% |
| | 514.97/LARS 414/MAFF 240422) (Cucumber anthracnose | |
| | fungus) (Colletotrichum lagenarium) | |
| G2RH83 | Thielavia terrestris (strain ATCC 38088/NRRL 8126) | 70.00% |
| | (Acremonium alabamense) | |
| G2QPF6 | Thielavia heterothallica (strain ATCC 42464/BCRC 31852/ | 71.00% |
| | DSM 1799) (Myceliophthora thermophila) | |
| M3B392 | Mycosphaerella fijiensis (strain CIRAD86) (Black leaf streak | 71.00% |
| | disease fungus) (Pseudocercospora fijiensis) | |
| J3PBB2 | Gaeumannomyces graminis var. tritici (strain R3-111a-1) (Wheat | 67.00% |
| | and barley take-all root rot fungus) | |
| G2XBJ6 | Verticillium dahliae (strain VdLs.17/ATCC MYA-4575/FGSC | 68.00% |
| | 10137) (Verticillium wilt) | |
| C9SLB0 | Verticillium alfalfae (strain VaMs.102/ATCC MYA-4576/ | 68.00% |
| | FGSC 10136) (Verticillium wilt of alfalfa) (Verticillium albo- | |
| | atrum) | |
| L7JDG8 | Magnaporthe oryzae (strain P131) (Rice blast fungus) (Pyricularia | 67.00% |
| | oryzae) | |
| L7HXX7 | Magnaporthe oryzae (strain Y34) (Rice blast fungus) (Pyricularia | 67.00% |
| | oryzae) | |
| G4MRF2 | Magnaporthe oryzae (strain 70-15/ATCC MYA-4617/FGSC | 67.00% |
| | 8958) (Rice blast fungus) (Pyricularia oryzae) | |
| F0X7H1 | Grosmannia clavigera (strain kw1407/UAMH 11150) (Blue | 70.00% |
| | stain fungus) (Graphiocladiella clavigera) | |
| E5R4F7 | Leptosphaeria maculans (strain JN3/isolate v23.1.3/race Av1-4- | 69.00% |
| | 5-6-7-8) (Blackleg fungus) (Phoma lingam) | |
| M2NDS8 | Baudoinia compniacensis (strain UAMH 10762) (Angels' share | 71.00% |
| | fungus) | |
| R8BW20 | Togninia minima (strain UCR-PA7) (Esca disease fungus) | 66.00% |
| | (Phaeoacremonium aleophilum) | |
| U7PM18 | Sporothrix schenckii (strain ATCC 58251/de Perez 2211183) | 69.00% |
| | (Rose-picker's disease fungus) | |
| M3CXY4 | Sphaerulina musiva (strain SO2202) (Poplar stem canker fungus) | 64.00% |
| | (Septoria musiva) | |
| M4FJF4 | Magnaporthe poae (strain ATCC 64411/73-15) (Kentucky | 65.00% |
| | bluegrass fungus) | |
| Q2GVA6 | Chaetomium globosum (strain ATCC 6205/CBS 148.51/DSM | 69.00% |
| | 1962/NBRC 6347/NRRL 1970) (Soil fungus) | |
| W3WZP2 | Pestalotiopsis fici W106-1 | 62.00% |
| A7EZX1 | Sclerotinia sclerotiorum (strain ATCC 18683/1980/Ss-1) | 69.00% |
| | (White mold) (Whetzelinia sclerotiorum) | |
| R0K8K2 | Setosphaeria turcica (strain 28A) (Northern leaf blight fungus) | 68.00% |
| | (Exserohilum turcicum) | |
| S3C0P8 | Ophiostoma piceae (strain UAMH 11346) (Sap stain fungus) | 66.00% |
| G0RZ60 | Chaetomium thermophilum (strain DSM 1495/CBS 144.50/IMI | 72.00% |
| | 039719) | |
| W9XAR0 | Capronia epimyces CBS 606.96 | 66.00% |
| H6BU98 | Exophiala dermatitidis (strain ATCC 34100/CBS 525.76/ | 68.00% |
| | NIH/UT8656) (Black yeast) (Wangiella dermatitidis) | |
| N1PEF2 | Mycosphaerella pini (strain NZE10/CBS 128990) (Red band | 67.00% |
| | needle blight fungus) (Dothistroma septosporum) | |
| W9XE16 | Cladophialophora psammophila CBS 110553 | 68.00% |

TABLE 2
Homologous sequences to the membrane-bound T. reesei copper transporting
ATPase (or copper permease) (SEQ ID NO: 6). Table 2 shows the accession number
(UNIPROT), organism and sequence identity to SEQ ID NO: 6. The protein sequence database
UNIPROT was used as source of the amino acid sequences. Sequence identity was determined
using a standard protein-protein BLAST (blastp) against the Uniprot database on the
NCBI/BLAST website.

| | | % ID to T. reesei |
|---|---|---|
| Accession | | Copper |
| No. | | Exporting |
| (UNIPROT) | Organism/Strain | ATPase |
| G0RK31 | Hypocrea jecorina (strain QM6a) (Trichoderma reesei) | 100.00% |
| G9N254 | Hypocrea virens (strain Gv29-8/FGSC 10586) (Gliocladium | 84.00% |
| | virens) (Trichoderma virens) | |
| G9PAF2 | Hypocrea atroviridis (strain ATCC 20476/IMI 206040) | 75.00% |
| | (Trichoderma atroviride) | |
| E9ECM0 | Metarhizium acridum (strain CQMa 102) | 74.00% |
| E9EKQ2 | Metarhizium anisopliae (strain ARSEF 23/ATCC MYA- | 73.00% |
| | 3075) | |
| G3JK92 | Cordyceps militaris (strain CM01) (Caterpillar fungus) | 71.00% |
| J4WLH8 | Beauveria bassiana (strain ARSEF 2860) (White muscardine | 71.00% |
| | disease fungus) (Tritirachium shiotae) | |
| X0F5I6 | Fusarium oxysporum f. sp. radicis-lycopersici 26381 | 71.00% |
| W9L8T5 | Fusarium oxysporum Fo47 | 71.00% |
| X0IUR8 | Fusarium oxysporum f. sp. conglutinans race 2 54008 | 71.00% |
| F9F4A0 | Fusarium oxysporum (strain Fo5176) (Fusarium vascular | 71.00% |
| | wilt) | |
| S0DI52 | Gibberella fujikuroi (strain CBS 195.34/IMI 58289/NRRL | 71.00% |
| | A-6831) (Bakanae and foot rot disease fungus) (Fusarium | |
| | fujikuroi) | |
| X0ARP5 | Fusarium oxysporum f. sp. melonis 26406 | 71.00% |
| W9Q9P3 | Fusarium oxysporum f. sp. pisi HDV247 | 71.00% |
| N4UMC8 | Fusarium oxysporum f. sp. cubense (strain race 1) (Panama | 71.00% |
| | disease fungus) | |
| X0CHX5 | Fusarium oxysporum f. sp. raphani 54005 | 71.00% |
| W9M4Y1 | Fusarium oxysporum f. sp. lycopersici MN25 | 71.00% |
| W9HH20 | Fusarium oxysporum FOSC 3-a | 71.00% |
| X0K9C1 | Fusarium oxysporum f. sp. cubense tropical race 4 54006 | 71.00% |
| J9N7Q4 | Fusarium oxysporum f. sp. lycopersici (strain 4287/CBS | 71.00% |
| | 123668/FGSC 9935/NRRL 34936) (Fusarium vascular wilt | |
| | of tomato) | |
| X0N9B8 | Fusarium oxysporum f. sp. vasinfectum 25433 | 71.00% |
| N1RJG7 | Fusarium oxysporum f. sp. cubense (strain race 4) (Panama | 71.00% |
| | disease fungus) | |
| W7MRF0 | Gibberella moniliformis (strain M3125/FGSC 7600) (Maize | 70.00% |
| | ear and stalk rot fungus) (Fusarium verticillioides) | |
| C7YWD7 | Nectria haematococca (strain 77-13-4/ATCC MYA-4622/ | 71.00% |
| | FGSC 9596/MPVI) (Fusarium solani subsp. pisi) | |
| K3W0V9 | Fusarium pseudograminearum (strain CS3096) (Wheat and | 70.00% |
| | barley crown-rot fungus) | |
| M1WIK4 | Claviceps purpurea (strain 20.1) (Ergot fungus) (Sphacelia | 70.00% |
| | segetum) | |
| T0KKX9 | Colletotrichum gloeosporioides (strain Cg-14) (Anthracnose | 70.00% |
| | fungus) (Glomerella cingulata) | |
| Q0WXV8 | Glomerella lagenarium (Anthracnose fungus) (Colletotrichum | 70.00% |
| | lagenarium) | |
| N4UX28 | Colletotrichum orbiculare (strain 104-T/ATCC 96160/CBS | 70.00% |
| | 514.97/LARS 414/MAFF 240422) (Cucumber anthracnose | |
| | fungus) (Colletotrichum lagenarium) | |
| G2WT58 | Verticillium dahliae (strain VdLs.17/ATCC MYA-4575/ | 69.00% |
| | FGSC 10137) (Verticillium wilt) | |
| Q8J286 | Colletotrichum lindemuthianum (Bean anthracnose fungus) | 69.00% |
| | (Glomerella lindemuthiana) | |
| H1UZ58 | Colletotrichum higginsianum (strain IMI 349063) (Crucifer | 70.00% |
| | anthracnose fungus) | |
| E3QAD8 | Colletotrichum graminicola (strain M1.001/M2/FGSC | 70.00% |
| | 10212) (Maize anthracnose fungus) (Glomerella graminicola) | |
| X0G9A8 | Fusarium oxysporum f. sp. radicis-lycopersici 26381 | 71.00% |
| W9L5N1 | Fusarium oxysporum Fo47 | 71.00% |
| G4N6G7 | Magnaporthe oryzae (strain 70-15/ATCC MYA-4617/ | 69.00% |
| | FGSC 8958) (Rice blast fungus) (Pyricularia oryzae) | |
| X0IFU3 | Fusarium oxysporum f. sp. conglutinans race 2 54008 | 72.00% |
| X0ASZ2 | Fusarium oxysporum f. sp. melonis 26406 | 71.00% |
| W9QGK7 | Fusarium oxysporum f. sp. pisi HDV247 | 71.00% |
| X0DH57 | Fusarium oxysporum f. sp. raphani 54005 | 71.00% |
| W9MAB3 | Fusarium oxysporum f. sp. lycopersici MN25 | 71.00% |
| W9HH28 | Fusarium oxysporum FOSC 3-a | 71.00% |
| X0M7A2 | Fusarium oxysporum f. sp. vasinfectum 25433 | 71.00% |
| L7JFD3 | Magnaporthe oryzae (strain P131) (Rice blast fungus) | 69.00% |
| | (Pyricularia oryzae) | |
| L7I603 | Magnaporthe oryzae (strain Y34) (Rice blast fungus) | 69.00% |
| | (Pyricularia oryzae) | |
| G2REL9 | Thielavia terrestris (strain ATCC 38088/NRRL 8126) | 69.00% |
| | (Acremonium alabamense) | |
| W3WMU8 | Pestalotiopsis fici W106-1 | 68.00% |
| B2AAH3 | Podospora anserina (strain S/ATCC MYA-4624/DSM 980/ | 69.00% |
| | FGSC 10383) (Pleurage anserina) | |
| C9SH44 | Verticillium alfalfae (strain VaMs.102/ATCC MYA-4576/ | 68.00% |
| | FGSC 10136) (Verticillium wilt of alfalfa) (Verticillium albo- | |
| | atrum) | |
| M4G378 | Magnaporthe poae (strain ATCC 64411/73-15) (Kentucky | 68.00% |
| | bluegrass fungus) | |
| R8BNC2 | Togninia minima (strain UCR-PA7) (Esca disease fungus) | 69.00% |
| | (Phaeoacremonium aleophilum) | |

TABLE 3
Examples of cuproenzymes originally classified as glycoside hydrolases 61
(GH61) family and now classified as AA9 (copper-dependent lytic polysaccharide
monooxygenases (LPMOs)).

| Organism | GenBank Accession Nos. | Uniprot Nos. |
|---|---|---|
| Agaricus bisporus | AAA53434.1 | Q00023 |
| Aspergillus fumigatus | CAF31975.1, AFJ54163.1 | Q6MYM8, |
| Aspergillus kawachii | BAB62318.1 | Q96WQ9 |
| Aspergillus nidulans | EAA65609.1, EAA59072.1, EAA66740.1, | C8VTW9, Q5BEI9, |
| | CBF83171.1, EAA59545.1, EAA58450.1, | Q5B7G9, C8VI93, |
| | EAA63617.1, EAA59125.1, EAA64722.1, | Q5AQA6, Q5AUY9, |
| | ABF50850.1, EAA64499.1 | C8V0F9, Q5AZ52, |
| | | C8VIS7, Q5B8T4, |
| | | C8V6H2, Q5B6H0, |
| | | Q5BCX8, C8VNP4, |
| | | Q5BAP2 |
| Aspergillus niger | CAK38942.1, CAK45495.1, CAK41095.1, | A2QJX0, A2QR94, |
| | CAK97151.1, CAK46515.1, CAK97324.1, | A2QYU6, A2QZE1, |
| | CAK42466.1 | A2R313, A2R5J9, |
| | | A2R5N0 |
| Aspergillus oryzae | BAE55582.1, BAE56764.1, BAE58643.1, | Q2US83, Q2UNV1, |
| | BAE58735.1, BAE59290.1, BAE60320.1, | Q2UIH2, Q2UI80, |
| | BAE64395.1, BAE65561.1 | Q2UGM5, Q2UDP5, |
| | | Q2U220, Q2TYW2 |
| Bipolaris maydis | AAM76663.1 | Q8J0H7 |
| Botryotinia fuckeliana | CCD34368.1, CCD47228.1, CCD48549.1, | |
| | CCD50139.1, CCD50144.1, CCD51504.1, | |
| | CCD49290.1, CCD52645.1, CCD50451.2, | |
| | CCD50451.1 | |
| Chaetomium | AGY80102.1, AGY80103.1, AGY80104.1, | |
| thermophilum | AGY80105.1, AGY80103.1, AGY80104.1, | |
| | AGY80105.1 | |
| Colletotrichum | CAQ16278.1, CAQ16206.1, CAQ16208.1, | B5WYD8, B5WY66, |
| graminicola | CAQ16217.1 | B5WY68, B5WY77 |
| Coprinopsis cinerea | CAG27578.1 | |
| Cryptococcus bacillisporus | ADV19810.1 | |
| Cryptococcus neoformans | AFR92731.1, AFR92731.2, AAC39449.1, | O59899, F5HH24 |
| | AAW41121.1 | |
| Flammulina velutipes | ADX07320.1 | |
| Fusarium fujikuroi | CCT72465.1, CCT67119.1, CCT69268.1, | |
| | CCT72729.1, CCT72942.1, CCT73805.1, | |
| | CCT74544.1, CCT74587.1, CCT67584.1, | |
| | CCT75380.1, CCT67584.1, CCT75380.1, | |
| | CCT64153.1, CCT64954.1, CCT63889.1 | |
| Fusarium graminearum | ABT35335.1, XP_383871.1 | |
| Gloeophyllum trabeum | AEJ35168.1 | |
| Heterobasidion | AFO72234.1, AFO72233.1, AFO72232.1, | |
| parviporum | AFO72235.1, AFO72236.1, AFO72237.1, | |
| | AFO72238.1, AFO72239.1 | |
| Humicola insolens | CAG27577.1 | |
| Hypocrea orientalis | AFD50197.1 | |
| Lasiodiplodia theobromae | CAJ81215.1, CAJ81216.1, CAJ81217.1, | |
| | CAJ81218.1 | |
| Leptosphaeria maculans | CBX91313.1, CBX93546.1, CBX94224.1, | E4ZJM8, E4ZQ11, |
| | CBX94532.1, CBX94572.1, CBX95655.1, | E4ZS44, E4ZSU4, |
| | CBX96476.1, CBX96550.1, CBX96949.1, | E4ZSY4, E4ZVM9, |
| | CBX97718.1, CBX98126.1, CBY01974.1, | E4ZZ41, E4ZYM4, |
| | CBY02242.1, CBX91667.1, CBX93965.1, | E5A089, E5A201, |
| | CBX98254.1, CBY00196.1, CBY01204.1, | E5A3B3, E5AFI5, |
| | CBY01256.1, CBY01257.1 | E5ACP0, E4ZK72, |
| | | E4ZQA3, E5A3P1, |
| | | E5A955, E5AC13, |
| | | E5ADG7, E5ADG8 |
| Leucoagaricus | CDJ79823.1 | |
| gongylophorus | | |
| Magnaporthe grisea | EAA54572.1, XP_359989.1, EAA53409.1, | G4N3E5, G4MUY8, |
| | XP_367775.1, EAA56945.1, XP_367375.1, | G4MXC7, G4MXS5, |
| | EAA53298.1, XP_367664.1, EAA57051.1, | G4MS66, G4MVX4, |
| | XP_362437.1, EAA54517.1, XP_365800.1, | G4NAI5, G4N560, |
| | EAA57285.1, XP_362794.1, EAA57097.1, | G4NHT8, G4N2Z0, |
| | XP_362483.1, EAA50788.1, XP_362102.1, | |
| | EAA57439.1, XP_362640.1, EAA49718.1, | |
| | XP_364864.1, EAA50298.1, XP_361583.1, | |
| | EAA52941.1, XP_369395.1, EAA51422.1, | |
| | EAA56258.1, XP_369714.1, EAA53354.1, | |
| | XP_367720.1, XP_370106.1 | |
| Malbranchea cinnamomea | CCP37674.1 | |
| Melanocarpus albomyces | CCP37668.1 | |
| Myceliophthora fergusii | CCP37667.1 | |
| Myceliophthora | AEO61257.1, AEO56016.1, AEO54509.1, | |
| thermophila | AEO55082.1, AEO55652.1, AEO55776.1, | |
| | AEO56416.1, AEO56542.1, AEO56547.1, | |
| | AEO56642.1, AEO56665.1, AEO58412.1, | |
| | AEO58921.1, AEO59482.1, AEO59823.1, | |
| | AEO59836.1, AEO59955.1, AEO60271.1, | |
| | AEO61304.1, AEO61305.1, AEO56498.1, | |
| | AEO58169.1 | |
| Neurospora crassa | CAD21296.1, XP_326543.1, EAA32426.1, | Q1K8B6, Q8WZQ2, |
| | CAD70347.1, EAA26656.1, XP_322586.1, | Q1K4Q1, Q873G1, |
| | CAE81966.1, EAA36262.1, XP_329057.1, | Q7SHD9, Q7S411, |
| | CAF05857.1, EAA30230.1, XP_331120.1, | Q7SA19, Q7S1V2, |
| | EAA33178.1, XP_328604.1, EAA29347.1, | Q7SHI8, Q7S111, |
| | XP_325824.1, EAA36362.1, XP_330104.1, | Q7S1A0, Q7S439, |
| | EAA29018.1, XP_328466.1, EAA29132.1, | Q7SCJ5, Q7RWN7, |
| | XP_327806.1, EAA30263.1, XP_331016.1, | Q7SAR4, Q7RV41, |
| | EAA34466.1, XP_325016.1, EAA26873.1, | Q9P3R7 |
| | XP_330877.1, EAA33408.1, XP_328680.1, | |
| | EAA36150.1, CAB97283.2, XP_330187.1 | |
| Penicillium chrysogenum | CAP80988.1, CAP91809.1, CAP92380.1, | B6H016, B6H3U0, |
| | CAP86439.1 | B6H3A3, B6HG02 |
| Phanerochaete | AAM22493.1, BAL43430.1 | Q8NJI9 |
| chrysosporium | | |
| Piriformospora indica | CCA67659.1, CCA68244.1, CCA70035.1, | |
| | CCA70418.1, CCA70703.1, CCA72182.1, | |
| | CCA72183.1, CCA72192.1, CCA72220.1, | |
| | CCA73144.1, CCA73151.1, CCA74246.1, | |
| | CCA74814.1, CCA75037.1, CCA66803.1, | |
| | CCA67656.1, CCA67657.1, CCA67658.1, | |
| | CCA70417.1, CCA71764.1, CCA72221.1, | |
| | CCA74449.1, CCA76320.1, CCA76671.1, | |
| | CCA77877.1 | |
| Podospora anserina | CAP59702.1, CAP61395.1, CAP61476.1, | B2A9F5, B2AD80, |
| | CAP61650.1, CAP64619.1, CAP64732.1, | B2ADG1, B2ADY5, |
| | CAP64865.1, CAP65111.1, CAP65855.1, | B2AKU6, B2AL94, |
| | CAP65866.1, CAP65971.1, CAP66744.1, | B2ALM7, B2AMI8, |
| | CAP67176.1, CAP67190.1, CAP67201.1, | B2APD8, B2APE9, |
| | CAP67466.1, CAP67481.1, CAP67493.1, | B2API9, B2ARG6, |
| | CAP67740.1, CAP68173.1, CAP68309.1, | B2AS05, B2AS19, |
| | CAP68352.1, CAP68375.1, CAP71532.1, | B2AS30, B2ASU3, |
| | CAP71839.1, CAP72740.1, CAP73072.1, | B2ASV8, B2ASX0, |
| | CAP73254.1, CAP73311.1, CAP73320.1, | B2ATL7, B2AUV0, |
| | CAP61048.1, CAP70156.1, CAP70248.1 | B2AV86, B2AVC8, |
| | | B2AVF1, B2B346, |
| | | B2B403, B2B4L5, |
| | | B2B5J7, B2B629, |
| | | B2B686, B2B695, |
| | | B2AC83, B2AZV6, |
| | | B2AZD4 |
| Pyrenochaeta lycopersici | AEV53599.1 | |
| Rasamsonia | CCP37669.1 | |
| byssochlamydoides | | |
| Remersonia thermophila | CCP37675.1 | |
| Scytalidium indonesiacum | CCP37676.1 | |
| Sordaria macrospora k- | CAQ58424.1 | C1KU36 |
| hell | | |
| Thermoascus aurantiacus | ABW56451.1, ACS05720.1, CCP37673.1, | |
| | AGO68294.1 | |
| Thermomyces dupontii | CCP37672.1 | |
| Thermomyces lanuginosus | CCP37678.1 | |
| Thielavia terrestris | CAG27576.1, AEO62422.1, AEO67662.1, | |
| | AEO64605.1, AEO69044.1, AEO64177.1, | |
| | AEO64593.1, AEO65532.1, AEO65580.1, | |
| | AEO66274.1, AEO67396.1, AEO68023.1, | |
| | AEO68157.1, AEO68577.1, AEO68763.1, | |
| | AEO71031.1, AEO67395.1, AEO69043.1, | |
| | ACE10231.1, ACE10232.1, ACE10232.1, | |
| | ACE10233.1, ACE10233.1, AEO71030.1, | |
| | ACE10234.1, ACE10235.1, ACE10235.1 | |
| Trichoderma reesei | AAP57753.1, ABH82048.1, ACK19226.1, | Q7Z9M7, O14405 |
| | ACR92640.1, CAA71999.1 | |
| Trichoderma | ADB89217.1 | D3JTC4 |
| saturnisporum | | |
| Trichoderma sp. | ACH92573.1 | B5TYI4 |
| Trichoderma viride | ACD36971.1, ADJ57703.1, ACD36973.1 | B4YEW1, B4YEW3, |
| | | D9IXC6 |
| uncultured eukaryote | CCA94933.1, CCA94930.1, CCA94931.1, | |
| | CCA94932.1, CCA94934.1 | |
| Volvariella volvacea | AFP23133.1, AAT64005.1 | Q6E5B4 |
| Zea mays | ACF86151.1, ACF78974.1, ACR36748.1 | B4FA31 |

### Utility

The compositions and methods detailed herein provide numerous benefits to the production of cuproenzymes. For example, aspects of the present disclosure allow improved production of cuproenzymes used in industrial contexts, including cuproenzymes used in cellulosic biomass processing for the production of commercially relevant products, e.g., cellulosic ethanol. Improvements in the production of other cuproenzymes, e.g., laccases and tyrosinases, is also of clear commercial value (e.g., for uses in detergent, biofuel, and food applications).

Additionally, the compositions and methods of the present disclosure allow for a reduction in the total amount of copper employed in cuproenzyme production, which reduces the level of copper in waste water from the fermentation process, thus aiding in meeting regulatory requirements for this metal in industrial plant discharges.

Other aspects and embodiments of the present compositions and methods will be apparent from the foregoing description and following examples.

### Examples

Aspects of the present teachings may be further understood in light of the Examples, which should not be construed as limiting the present teachings in any way.

### Example 1: Effect of Copper on Tyrosinase Expressing Cells

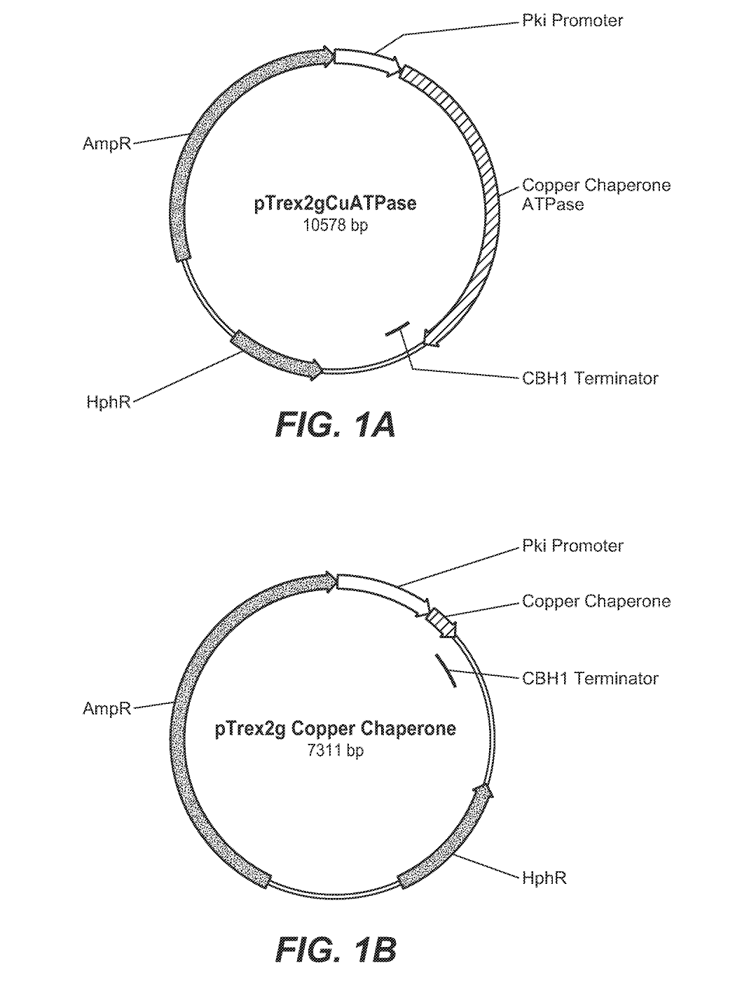

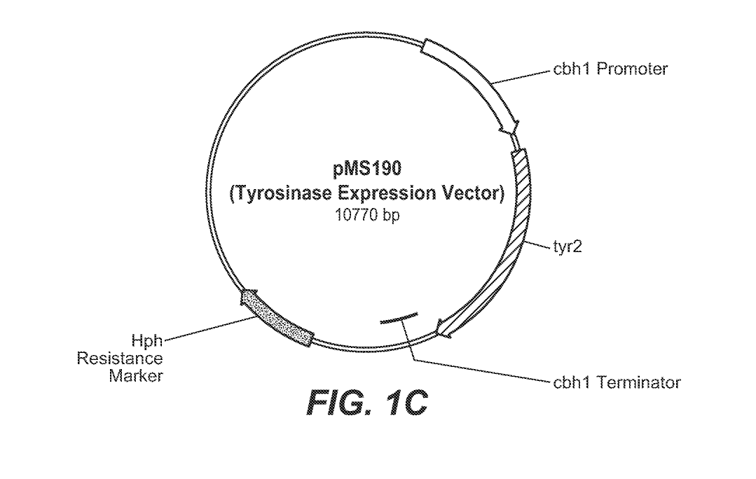

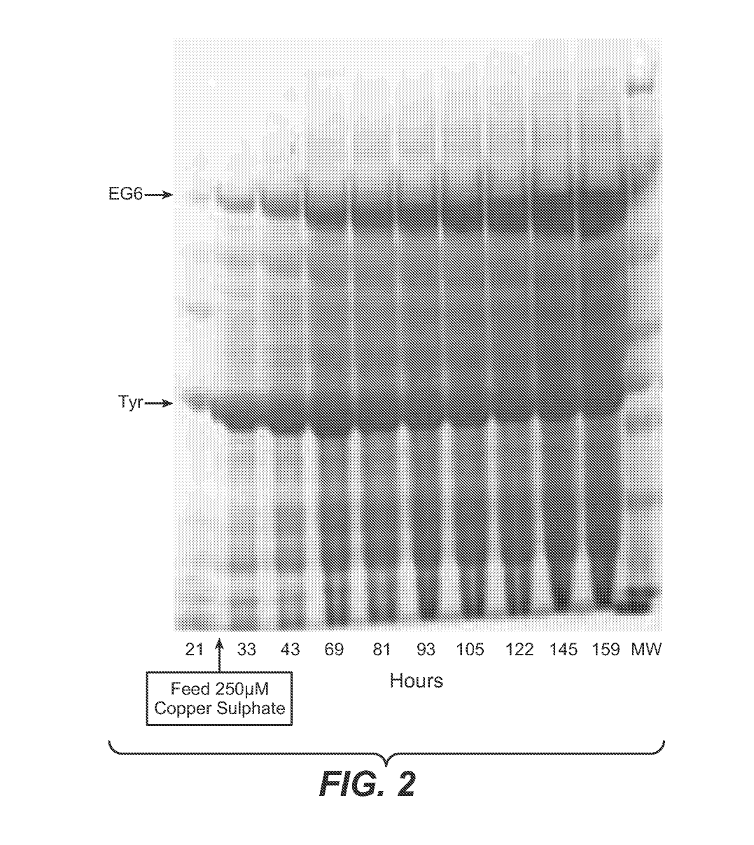

An expression vector for over-expressing T. reesei tyrosinase (SEQ ID NO:9) was generated (FIG. 1C) and transformed into a T. reesei host cell. The promoter driving the expression of the DNA sequence encoding T. reesei tyrosinase was the cbh1 promoter. The expression level of secreted proteins from these transformed host cells was determined in 14-L fermentation cultures. The cells were pre-grown in a flask with shaking at 34° C. and pH 3.5 until glucose was depleted. A glucose/sophorose feed was started and the temperature was shifted from 34° C. to 28° C. and the pH was shifted from 3.5 to 4. (Glucose/sophrose is an inducer of the cbh1 promoter). Dissolved oxygen % was kept constant by adjusting agitation, pressure and airflow. The fermentation was allowed to go for about 200 hours (depending on the rate of enzyme production). In FIG. 2, extracellular protein expression from the 14-L scale fermentation of the tyrosinase-expressing host cell above was analyzed by SDS-PAGE. Cultivation time is shown at the bottom in hours and the beginning of the copper feed during the fermentation is indicated with an upward arrow. The bands on the gel for the secreted enzymes tyrosinase and endoglucanase 6 are indicated at the left (Tyr and EG6, respectively). The copper-containing tyrosinase enzyme showed a peak production within 69 hours and then demonstrated decreased accumulation during the remaining time course. In contrast, the non-copper containing enzyme endoglucanase 6 (EG6) showed increasing accumulation over the entire time course. This demonstrates that copper containing enzymes were expressed less efficiently over time than non-copper containing enzymes.

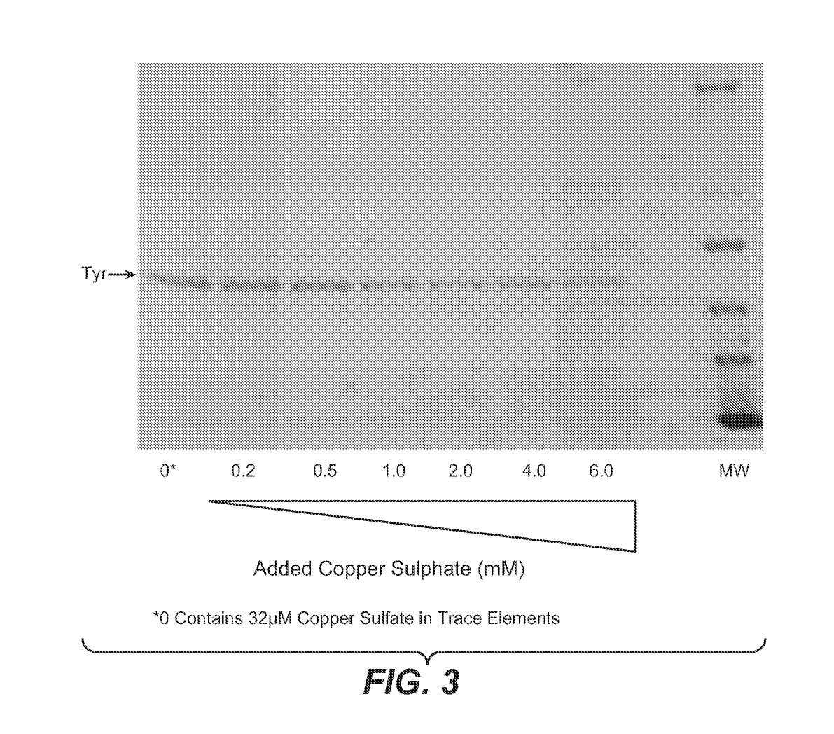

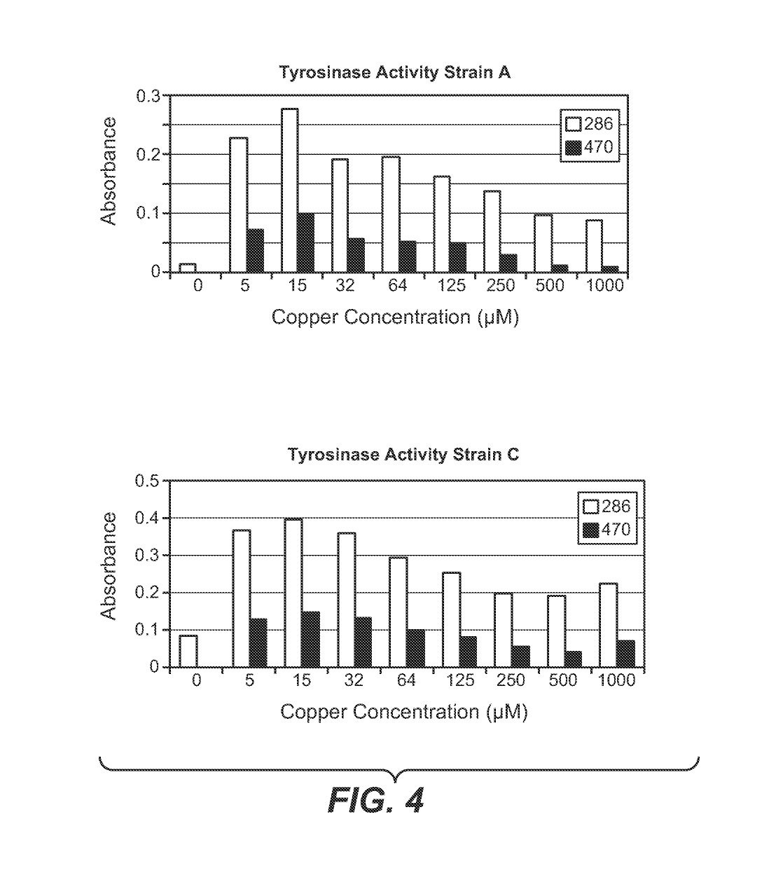

In an attempt to improve tyrosinase expression, the host cells over-expressing tyrosinase were cultured in different amounts of copper. FIG. 3 shows SDS-PAGE analysis of the expression of tyrosinase (Tyr) in the presence of increasing amounts of copper (shown at the bottom of each lane). As seen in this figure, increasing the amount of copper sulphate present in the growth media resulted in decreased production of tyrosinase, rather than increased production, from the host cell. This pattern was confirmed in assays of tyrosinase activity from two independent strains of host cells overexpressing tyrosinase (FIG. 4). In FIG. 4, tyrosinase over-expressing Strains A and C (top panel and bottom panel, respectively) were cultivated at different copper concentrations ranging from 0 to 1000 μM and tyrosinase activity in the culture supernatant was measured using tyrosine as substrate and detecting the formation of product at 286 nm (open bars) and 470 nm (filled bars). The highest concentration of copper that did not lead to adverse effect to protein production is approximately 15 μM. It was hypothesized that the additional copper was not being properly trafficked to the secretory pathway and thus leading to low tyrosinase secretion and/or cell toxicity.

### Example 2: Overexpression of Copper Metallochaperones Increases Tyrosinase Expression

Synthetic genes for the soluble copper transporter and membrane-bound copper transporting ATPase from T. reesei were identified by homology to known sequences and then synthesized (GeneArt®, Life Technologies). Expression vectors for these two T. reesei copper metallochaperones were constructed and employed to determine whether their over-expression could improve tyrosinase expression in the host cells of Example 1. FIGS. 1A-1B show schematics of (1A) the expression construct for the membrane-bound copper transporting ATPase and (1B) the expression construct for the cytoplasmic (soluble) copper transporter. These copper chaperone genes were expressed using the constitutive pyruvate kinase (pki) promoter and included a terminator derived from the CBH1 gene.

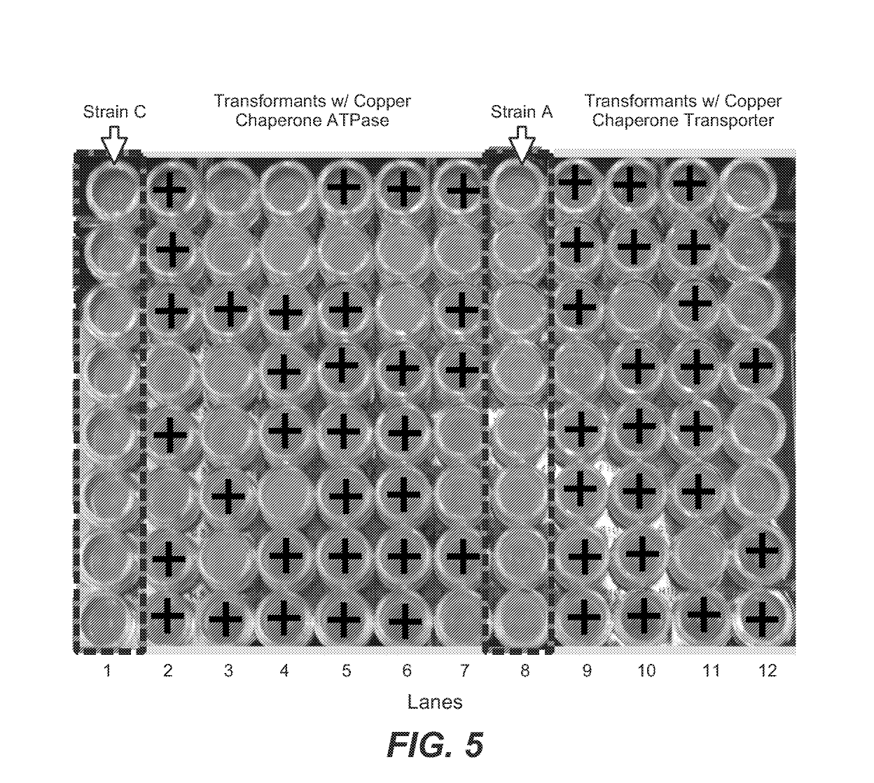

FIG. 5 shows the results of a spot assay for tyrosinase activity derived from tyrosinase overexpressing cells cultured in the presence of levels of copper that lead to reduced/undetectable tyrosinase expression (6 mM). Tyrosinase activity was detected in this assay by combining 10 μM of culture supernatant and 200 μM of 10% skim milk (pre-heated to 35° C.) in a microtiter plate and inclubating the mixture for 10 minutes (or longer) at 35° C. The milk turned from white to red when tyrosinase was present and active. Plus signs indicate wells with significant red color.

As expected, no tyrosinase activity could be detected in the control Strains A (wells in lane 8) and C (wells in lane 1), outlined with dotted lines. The ability of Strains A and C to produce tyrosinase was restored, however, when these strains are retransformed with either the membrane-bound copper transporting ATPase (wells in lanes 2-7) or the cytoplasmic (soluble) copper transporter plasmid (wells in lanes 9-12). Thus, expression of either of these copper chaperones resulted in significantly increased expression of the tyrosinase cuproenzyme.

### Example 3: Overexpression of Copper Metallochaperones Increases Laccase Expression

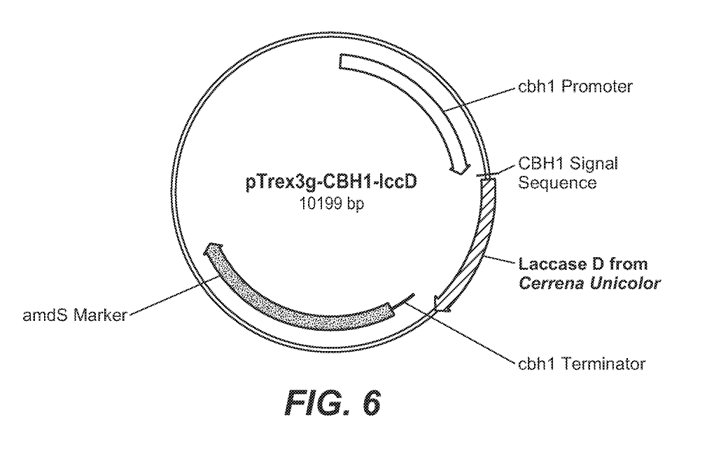

FIG. 6 shows an expression vector construct for the copper metalloprotein laccase D from Cerrena unicolor (transcribed from the cbh1 promoter with a CBH1 signal sequence and cbh1 transcriptional terminator). The mature laccase D sequence is SEQ ID NO: 10.

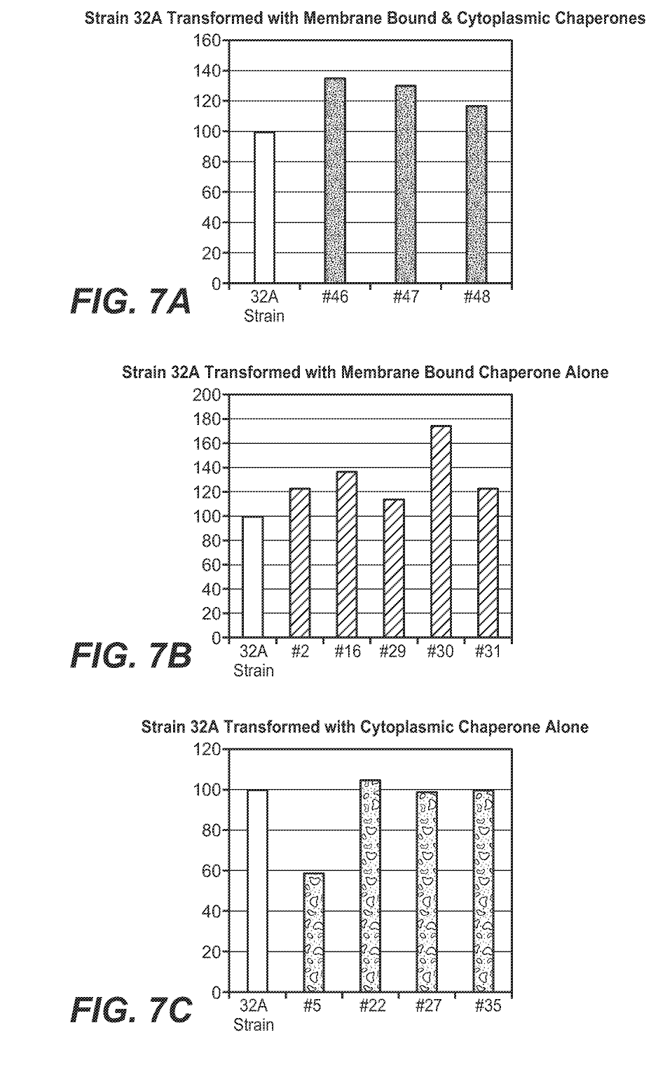

FIGS. 7A-7C show an analysis of laccase D production in a strain overexpressing laccase D (Strain 32A) both with and without over-expression of one or both of the copper metallochaperones described above (SEQ ID NOs: 3 and 6 expressed from the vectors which are depicted in FIG. 1). FIG. 7A shows relative expression levels of laccase D in Strain 32A (leftmost bar; set at 100%) and strains derived therefrom (#46, #47, and #48) which overexpressed both cytosolic transporter and membrane-bound copper transporting ATPase (transformed with the expression vectors shown in FIGS. 1A and 1B). FIG. 7B shows relative expression levels of laccase D in Strain 32A (leftmost bar; set at 100%) and strains derived therefrom (#2, #16, #29, #30 and #31) which overexpressed only the membrane-bound copper transporting ATPase (transformed with the expression vector shown in FIG. 1A). FIG. 7C shows relative expression levels of laccase D in Strain 32A (leftmost bar; set at 100%) and strains derived therefrom (#5, #22, #27 and #35) which overexpressed only the cytosolic copper transporter (transformed with the expression vector shown in FIG. 1B). The transformants were cultivated in microtiter plates for 5 days and laccase expression was determined using the ABTS assay (ABTS=2,2′-azino-bis(3-ethylberizothiazoline-6-sulphonic acid)). For the ABTS assay, 10 μL of 5-day liquid cultures were transferred to a new plate and 150 μL. 100 mM NaOAc, pH 5, and 20 μL. 4.5 mM ABTS were added. The OD₄₂₀ was measured using a Spectra Max spectrophotometer for 5 minutes at 20-second intervals. This data shows that expression of the membrane-bound copper transporter ATPase alone or in combination with the cytoplasmic (soluble) copper transporter significantly improved laccase D production.

Although the foregoing compositions and methods have been described in some detail by way of illustration and example for purposes of clarity of understanding, it is readily apparent to those of ordinary skill in the art in light of the teachings herein that certain changes and modifications may be made thereto without departing from the spirit or scope of the appended claims.

Accordingly, the preceding merely illustrates the principles of the present compositions and methods. It will be appreciated that those skilled in the art will be able to devise various arrangements which, although not explicitly described or shown herein, embody the principles of the present compositions and methods and are included within its spirit and scope. Furthermore, all examples and conditional language recited herein are principally intended to aid the reader in understanding the principles of the present compositions and methods and the concepts contributed by the inventors to furthering the art, and are to be construed as being without limitation to such specifically recited examples and conditions. Moreover, all statements herein reciting principles, aspects, and embodiments of the present compositions and methods as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present compositions and methods, therefore, is not intended to be limited to the exemplary embodiments shown and described herein.

List of Sequences

| SEQ ID | | |
|---|---|---|
| NO | Description | Sequence |
| 1 | gene sequence of T. | ATGTCTGAGACGCACACCTACGAGTTCAACGTCAC |
| | reesei cytoplasmic | CATGACCTGCGGCGGCTGCTCCGGCGCCATCGACC |
| | (soluble) copper | GAGTCCTCAAGAAGCTCGAGGGTACGTTCTTGAAC |
| | transporter | AATCATTCTCCCCTCTCCTCTCCTCTCCTCCCCTCTC |
| | | TCTCTCCCTCTCTCTCCTCCGTCATGCCGTAGGAGC |
| | | ACTGTCTGCGCCCCTCCCCCTCCAAAGAAAAACAC |
| | | AGCACTGACTCGGTTGGTTTTCTTTCTTTCTCGCAG |
| | | GCGTCGAAAGCTACGAAGTCTCCCTCGACAACCAG |
| | | ACCGCAAAGGTCGTCACCGCGCTGCCCTACGAGAC |
| | | GGTCCTGACCAAGATTGCCAAGACGGGCAAGAAG |
| | | ATCAACTCGGCGACGGCCGACGGCGTGCCGCAGTC |
| | | TGTCGAGGTATCTGTGTAG |
| 2 | coding sequence of | ATGTCTGAGACGCACACCTACGAGTTCAACGTCAC |
| | T. reesei | CATGACCTGCGGCGGCTGCTCCGGCGCCATCGACC |
| | cytoplasmic | GAGTCCTCAAGAAGCTCGAGGGGGCGTCGAAAGC |
| | (soluble) copper | TACGAAGTCTCCCTCGACAACCAGACCGCAAAGGT |
| | transporter | CGTCACCGCGCTGCCCTACGAGACGGTCCTGACCA |
| | (including stop | AGATTGCCAAGACGGGCAAGAAGATCAACTCGGC |
| | codon) | GACGGCCGACGGCGTGCCGCAGTCTGTCGAGGTAT |
| | | CTGTGTAG |
| 3 | amino acid | MSETHTYEFNVTMTCGGCSGAIDRVLKKLEGVESYE |
| | sequence of T. | VSLDNQTAKVVTALPYETVLTKIAKTGKKINSATAD |
| | reesei cytoplasmic | GVPQSVEVSV |
| | (soluble) copper | |
| | transporter | |
| 4 | gene sequence of T. | ATGGCCCCAACATACATCAAAGTCCCCGGGCGGG |
| | reesei membrane- | ACAATGATGAGCATGCGAGTGCGACCCTTACGCCA |
| | bound copper | AAGAGCGCGCACATGGCCACAACCACTCTGCGCGT |
| | transporting | TGGTGGCATGACGTAGGTTTCGTCCGTTTCCGGCT |
| | ATPase | GTGCTTCCGGCCAAGGTCTGCAGCACAAGCATGGC |
| | | TGGTCATTCTTTCTAACACTTCTTCTTGCAGATGTG |
| | | GTTCGTGCACAGCAGCCGTCGAGGGCGGCTTCAAG |
| | | GGCGTCAAGGGCGTTGGTACCGTCTCCGTCAGCCT |
| | | TGTTATGGAGAGGGCTGTCGTAATGCACGACCCCC |
| | | GGATCATCAGCGCTGAACAGGTTCGAGAGATTATC |
| | | GAAGATTGTGGATTCGACGCTGAGCTGCTGTCGAC |
| | | GGACCTCTTGAGCCCACTCGTCCCTCGATTCTCGG |
| | | ATGCCAAGGGGGATGAGGACATCGATAGCGGCCT |
| | | CTTGACGACCACGGTAGCCATCGAAGGCATGACGT |
| | | GTGGCGCCTGTACATCTGCTGTCGAGGGTGGATTC |
| | | AAGGATATCCCAGGTGTCAAGAGCTTCAGCATCTC |
| | | GCTTCTTTCTGAGCGAGCCGTCATCGAACACGATC |
| | | CAGAACTTTTGCCCACCGACAAGATTACCGAAATC |
| | | ATCGAAGACCGGGGCTTTGGTGCCGAAATCGTCGA |
| | | TTCCGTGAAGGCGCAACCTGGCAGCAGTACCGAG |
| | | GCTGAGAACCCAGCAAGTCATGTCGTGACTACGAC |
| | | GGTAGCCATCGAAGGAATGACTTGCGGTGCCTGTA |
| | | CGTCTGCTGTTGAGGGAGGCTTTCAGGGAGTTGAC |
| | | GGCATCCTGAAATTCAACATCAGTCTTCTGGCCGA |
| | | AAGGGCAGTCATTACTCACGATGTCACCAAGATCT |
| | | CCGCCGAACAGATTTCCGAAATCGTTGAAGACCGG |
| | | GGATTTGGTGCTACGGTTTTGTCCACCGTCCCGGA |
| | | GGCAAACGATCTCAGCAGTACGACCTCGCAGTTCA |
| | | AAATCTATGGCAGCCCGGACGCCGCCACTGCAAA |
| | | GGAGCTGGAGGAAAAGCTGCTGGCACTTGCTGGT |
| | | GTTAAATCTGCTTCCCTCAGCCTATCAACGGACCG |
| | | CCTGTCCGTCACGCACCAGCCTGCCGTCATTGGGC |
| | | TCCGAGGGATCGTCGAGGCGGTAGAGGCGCAAGG |
| | | CCTGAATGCTTTGGTGGCGGACAGCCACGACAACA |
| | | ACGCGCAACTCGAATCCTTGGCCAAGACTCGCGAG |
| | | ATCCAGGAATGGAGGACGGCGTGCAAGACGTCCG |
| | | CCTCGTTCGCCATTCCGGTATTCGTTCTTTCCATGG |
| | | TGTTGCCTATGATCTCAGACAGTCTGAACCTGAGT |
| | | CTAATCCACCTTGGCCATGGTCTCTACCTCGGCGA |
| | | CGTCGTCAACTTGGTACTCACAACACCTGTTCAGT |
| | | TTGGGGTTGGAAAGCGCTTTTACGTCTCGGCCTTC |
| | | AAGTCGCTCAAGCACCGTTCGCCGACTATGGATGT |
| | | GCTCGTCATGCTCGGCACCTCCTGCGCTTACTTCTT |
| | | CAGCATCTTCTCCATGGTCATCTCTATCCTCTTCGA |
| | | GCCTCATTCCCCGCCGGGCACGATCTTTGACACCA |
| | | GCACCATGCTCATCACCTTTGTGACCTTGGGCCGC |
| | | TATCTTGAGAACAGCGCCAAGGGTCAGACATCAA |
| | | AGGCTCTGTCCCGTCTCATGTCTCTAGCCCCGTCGA |
| | | TGGCCACCATCTACACGGATCCCATTGCCGCGGAG |
| | | AAGGCAGCAGAATCATGGGCCAAGTCAACCGATA |
| | | CACCCGCGGATGCGAAAGGCCAACCGTCTGGAGA |
| | | TGCGAGCGGCTCGTCGTACGAGGAGAAGAGCATC |
| | | CCTACTGAGCTGCTTCAGGTGGGAGATATCGTCGT |
| | | CATCCGACCCGGTGATAAGATTCCGGCGGACGGCG |
| | | TCGTTATGCGAGGAGAGACCTACGTCGACGAGAG |
| | | CATGGTCACCGGAGAGGCAATGCCGGTGCAGAAG |
| | | AGGATTGGCAGCAACGTGATTGGAGGCACGGTCA |
| | | ACGGCAACGGCAGAGTGGACTTTCGCGTCACCCGA |
| | | GCCGGGCGGGATACCCAGCTCAGTCAGATTGTCAA |
| | | GCTTGTTCAGGACGCGCAGACGACGAGGGCGCCT |
| | | ATTCAAAAGGTGGCCGACACTTTGGCTGGCTACTT |
| | | TGTGCCTACAATCTTGCTGCTCGGCATCCTCACCTT |
| | | CCTTGGCTGGTTGATCCTCAGCCACGCCCTGTCGC |
| | | ACCCCCCTATGATTTTCTTGAAGAACACCAGTGGT |
| | | GGCAAGGTCATGATTTGCGTCAAGCTGTGCATCTC |
| | | CGTCATTGTATTTGCATGCCCTTGTGCTCTGGGCCT |
| | | GGCCACGCCGACAGCTGTCATGGTAGGCACGGGC |
| | | GTGGGCGCTGAGAATGGCATCCTCATCAAAGGCG |
| | | GAGCTGCGCTGGAGCGAACCACCCAGGTTACCAA |
| | | AGTCGTCTTGGACAAAACCGGCACAATCACTCGTG |
| | | GCAAAATGGAGGTCGCCAAGAGCGGCCTTGTGTTT |
| | | CCCTGGAATGACAACGTGTCGCAGACCAAAGTCTG |
| | | GTGGGCCGCTGTCGGTCTGGCGGAAATGGGCAGC |
| | | GAGCACCCTATCGGAAGGGCGATTCTGGCAGCGG |
| | | CCAAGGCAGAAGTCGGCATCCTTGAAGCCGAAGC |
| | | CGCCATTCCAGGAAGCGTCAATGATTTCAAGTTGA |
| | | CTGTTGGCAAGGGCATCGATGCTATCGTTGAACCT |
| | | GCATTATCCGGTGATCGGACACGCTATAGGGTCCT |
| | | TGCTGGAAATGTCACCTTCCTTGAAGAGAACGGCG |
| | | TCGAGGTCCCCAAGGATGCCGTCGAGGCAGCAGA |
| | | GCGAATCAACTCGTCCGTCAAGAGCTCACGAGCCA |
| | | AGGCTGTGACTGCGGGCACGACCAACATCTTTGTC |
| | | GCCATTGATGGAAAGTACAGCGGCCACCTTTGTCT |
| | | CTCCGACACCATCAAAGATGGGGCGGCCGGGGTC |
| | | ATTTCTGTACTGCATAGCATGGGCATCAAGACGGC |
| | | CATGGTGACGGGAGACCAGCGACCCACCGCCCTG |
| | | GCCGTTGCCGCCCTCGTGGGCATCTCTCCCGAGGA |
| | | CGTGTTTGCCGGCGTCAGCCCCGACCAGAAGCAGG |
| | | TGATAGTACAGCAGTTCCAGAACCAGGGAGAGGT |
| | | GGTCGCCATGGTGGGAGACGGCATCAACGACTCG |
| | | CCGGCCCTCGCTACGGCCGACGTTGGTATCGCCAT |
| | | GTCGAGCGGAACGGACGTGGCCATGGAGGCCGCA |
| | | GATGTTGTGCTTATGCGTCCCGACGACCTGCTGAG |
| | | CATCCCGTCCGCCATCCACCTCACTCGGACCATCTT |
| | | CCGCCGCATCAAGCTGAACCTGGCGTGGGCATGCA |
| | | TCTACAACATTGTCGGCCTGCCCATTGCCATGGGT |
| | | TTCTTCCTGCCGTTTGGCATCCACATGCACCCCATG |
| | | TTCGCCGGGTTCGCCATGGCCTGCAGTAGCATTAG |
| | | TGTAGTGGTTAGCAGCCTGGCGCTCCGATGGTGGC |
| | | AACGACCGCAGTGGATGGACGAGGCGTCCGAACC |
| | | GGCGGGTGGCCTGCGCTGGATGAGCGGCACGGGC |
| | | ATCGTTGGCTGGGCTAAGGAGACGTTTGGACGCGT |
| | | CAGGAGAGGGAAGCGTGAGGAGGGTTACGTGGCG |
| | | TTGGAGAATTTAGAGGTCTGA |
| 5 | coding sequence of | ATGGCCCCAACATACATCAAAGTCCCCGGGCGGG |
| | T. reesei | ACAATGATGAGCATGCGAGTGCGACCCTTACGCCA |
| | membrane-bound | AAGAGCGCGCACATGGCCACAACCACTCTGCGCGT |
| | copper transporting | TGGTGGCATGACATGTGGTTCGTGCACAGCAGCCG |
| | ATPase | TCGAGGGCGGCTTCAAGGGCGTCAAGGGCGTTGGT |
| | | ACCGTCTCCGTCAGCCTTGTTATGGAGAGGGCTGT |
| | | CGTAATGCACGACCCCCGGATCATCAGCGCTGAAC |
| | | AGGTTCGAGAGATTATCGAAGATTGTGGATTCGAC |
| | | GCTGAGCTGCTGTCGACGGACCTCTTGAGCCCACT |
| | | CGTCCCTCGATTCTCGGATGCCAAGGGGGATGAGG |
| | | ACATCGATAGCGGCCTCTTGACGACCACGGTAGCC |
| | | ATCGAAGGCATGACGTGTGGCGCCTGTACATCTGC |
| | | TGTCGAGGGTGGATTCAAGGATATCCCAGGTGTCA |
| | | AGAGCTTCAGCATCTCGCTTCTTTCTGAGCGAGCC |
| | | GTCATCGAACACGATCCAGAACTTTTGCCCACCGA |
| | | CAAGATTACCGAAATCATCGAAGACCGGGGCTTTG |
| | | GTGCCGAAATCGTCGATTCCGTGAAGGCGCAACCT |
| | | GGCAGCAGTACCGAGGCTGAGAACCCAGCAAGTC |
| | | ATGTCGTGACTACGACGGTAGCCATCGAAGGAATG |
| | | ACTTGCGGTGCCTGTACGTCTGCTGTTGAGGGAGG |
| | | CTTTCAGGGAGTTGACGGCATCCTGAAATTCAACA |
| | | TCAGTCTTCTGGCCGAAAGGGCAGTCATTACTCAC |
| | | GATGTCACCAAGATCTCCGCCGAACAGATTTCCGA |
| | | AATCGTTGAAGACCGGGGATTTGGTGCTACGGTTT |
| | | TGTCCACCGTCCCGGAGGCAAACGATCTCAGCAGT |
| | | ACGACCTCGCAGTTCAAAATCTATGGCAGCCCGGA |
| | | CGCCGCCACTGCAAAGGAGCTGGAGGAAAAGCTG |
| | | CTGGCACTTGCTGGTGTTAAATCTGCTTCCCTCAGC |
| | | CTATCAACGGACCGCCTGTCCGTCACGCACCAGCC |
| | | TGCCGTCATTGGGCTCCGAGGGATCGTCGAGGCGG |
| | | TAGAGGCGCAAGGCCTGAATGCTTTGGTGGCGGAC |
| | | AGCCACGACAACAACGCGCAACTCGAATCCTTGGC |
| | | CAAGACTCGCGAGATCCAGGAATGGAGGACGGCG |
| | | TGCAAGACGTCCGCCTCGTTCGCCATTCCGGTATT |
| | | CGTTCTTTCCATGGTGTTGCCTATGATCTCAGACAG |
| | | TCTGAACCTGAGTCTAATCCACCTTGGCCATGGTC |
| | | TCTACCTCGGCGACGTCGTCAACTTGGTACTCACA |
| | | ACACCTGTTCAGTTTGGGGTTGGAAAGCGCTTTTA |
| | | CGTCTCGGCCTTCAAGTCGCTCAAGCACCGTTCGC |
| | | CGACTATGGATGTGCTCGTCATGCTCGGCACCTCC |
| | | TGCGCTTACTTCTTCAGCATCTTCTCCATGGTCATC |
| | | TCTATCCTCTTCGAGCCTCATTCCCCGCCGGGCACG |
| | | ATCTTTGACACCAGCACCATGCTCATCACCTTTGTG |
| | | ACCTTGGGCCGCTATCTTGAGAACAGCGCCAAGGG |
| | | TCAGACATCAAAGGCTCTGTCCCGTCTCATGTCTCT |
| | | AGCCCCGTCGATGGCCACCATCTACACGGATCCCA |
| | | TTGCCGCGGAGAAGGCAGCAGAATCATGGGCCAA |
| | | GTCAACCGATACACCCGCGGATGCGAAAGGCCAA |
| | | CCGTCTGGAGATGCGAGCGGCTCGTCGTACGAGGA |
| | | GAAGAGCATCCCTACTGAGCTGCTTCAGGTGGGAG |
| | | ATATCGTCGTCATCCGACCCGGTGATAAGATTCCG |
| | | GCGGACGGCGTCGTTATGCGAGGAGAGACCTACG |
| | | TCGACGAGAGCATGGTCACCGGAGAGGCAATGCC |
| | | GGTGCAGAAGAGGATTGGCAGCAACGTGATTGGA |
| | | GGCACGGTCAACGGCAACGGCAGAGTGGACTTTC |
| | | GCGTCACCCGAGCCGGGCGGGATACCCAGCTCAGT |
| | | CAGATTGTCAAGCTTGTTCAGGACGCGCAGACGAC |
| | | GAGGGCGCCTATTCAAAAGGTGGCCGACACTTTGG |
| | | CTGGCTACTTTGTGCCTACAATCTTGCTGCTCGGCA |
| | | TCCTCACCTTCCTTGGCTGGTTGATCCTCAGCCACG |
| | | CCCTGTCGCACCCCCCTATGATTTTCTTGAAGAAC |
| | | ACCAGTGGTGGCAAGGTCATGATTTGCGTCAAGCT |
| | | GTGCATCTCCGTCATTGTATTTGCATGCCCTTGTGC |
| | | TCTGGGCCTGGCCACGCCGACAGCTGTCATGGTAG |
| | | GCACGGGCGTGGGCGCTGAGAATGGCATCCTCATC |
| | | AAAGGCGGAGCTGCGCTGGAGCGAACCACCCAGG |
| | | TTACCAAAGTCGTCTTGGACAAAACCGGCACAATC |
| | | ACTCGTGGCAAAATGGAGGTCGCCAAGAGCGGCC |
| | | TTGTGTTTCCCTGGAATGACAACGTGTCGCAGACC |
| | | AAAGTCTGGTGGGCCGCTGTCGGTCTGGCGGAAAT |
| | | GGGCAGCGAGCACCCTATCGGAAGGGCGATTCTG |
| | | GCAGCGGCCAAGGCAGAAGTCGGCATCCTTGAAG |
| | | CCGAAGCCGCCATTCCAGGAAGCGTCAATGATTTC |
| | | AAGTTGACTGTTGGCAAGGGCATCGATGCTATCGT |
| | | TGAACCTGCATTATCCGGTGATCGGACACGCTATA |
| | | GGGTCCTTGCTGGAAATGTCACCTTCCTTGAAGAG |
| | | AACGGCGTCGAGGTCCCCAAGGATGCCGTCGAGG |
| | | CAGCAGAGCGAATCAACTCGTCCGTCAAGAGCTCA |
| | | CGAGCCAAGGCTGTGACTGCGGGCACGACCAACA |
| | | TCTTTGTCGCCATTGATGGAAAGTACAGCGGCCAC |
| | | CTTTGTCTCTCCGACACCATCAAAGATGGGGCGGC |
| | | CGGGGTCATTTCTGTACTGCATAGCATGGGCATCA |
| | | AGACGGCCATGGTGACGGGAGACCAGCGACCCAC |
| | | CGCCCTGGCCGTTGCCGCCCTCGTGGGCATCTCTC |
| | | CCGAGGACGTGTTTGCCGGCGTCAGCCCCGACCAG |
| | | AAGCAGGTGATAGTACAGCAGTTCCAGAACCAGG |
| | | GAGAGGTGGTCGCCATGGTGGGAGACGGCATCAA |
| | | CGACTCGCCGGCCCTCGCTACGGCCGACGTTGGTA |
| | | TCGCCATGTCGAGCGGAACGGACGTGGCCATGGA |
| | | GGCCGCAGATGTTGTGCTTATGCGTCCCGACGACC |
| | | TGCTGAGCATCCCGTCCGCCATCCACCTCACTCGG |
| | | ACCATCTTCCGCCGCATCAAGCTGAACCTGGCGTG |
| | | GGCATGCATCTACAACATTGTCGGCCTGCCCATTG |
| | | CCATGGGTTTCTTCCTGCCGTTTGGCATCCACATGC |
| | | ACCCCATGTTCGCCGGGTTCGCCATGGCCTGCAGT |
| | | AGCATTAGTGTAGTGGTTAGCAGCCTGGCGCTCCG |
| | | ATGGTGGCAACGACCGCAGTGGATGGACGAGGCG |
| | | TCCGAACCGGCGGGTGGCCTGCGCTGGATGAGCG |
| | | GCACGGGCATCGTTGGCTGGGCTAAGGAGACGTTT |
| | | GGACGCGTCAGGAGAGGGAAGCGTGAGGAGGGTT |
| | | ACGTGGCGTTGGAGAATTTAGAGGTCTGA |
| 6 | amino acid | MAPTYIKVPGRDNDEHASATLTPKSAHMATTTLRVG |
| | sequence of T. | GMTCGSCTAAVEGGFKGVKGVGTVSVSLVMERAVV |
| | reesei membrane- | MHDPRIISAEQVREIIEDCGFDAELLSTDLLSPLVPRFS |
| | bound copper | DAKGDEDIDSGLLTTTVAIEGMTCGACTSAVEGGFK |
| | transporting | DIPGVKSFSISLLSERAVIEHDPELLPTDKITEIIEDRGF |
| | ATPase | GAEIVDSVKAQPGSSTEAENPASHVVTTTVAIEGMTC |
| | | GACTSAVEGGFQGVDGILKFNISLLAERAVITHDVTK |
| | | ISAEQISEIVEDRGFGATVLSTVPEANDLSSTTSQFKIY |
| | | GSPDAATAKELEEKLLALAGVKSASLSLSTDRLSVTH |
| | | QPAVIGLRGIVEAVEAQGLNALVADSHDNNAQLESL |
| | | AKTREIQEWRTACKTSASFAIPVFVLSMVLPMISDSL |
| | | NLSLIHLGHGLYLGDVVNLVLTTPVQFGVGKRFYVS |
| | | AFKSLKHRSPTMDVLVMLGTSCAYFFSIFSMVISILFE |
| | | PHSPPGTIFDTSTMLITFVTLGRYLENSAKGQTSKALS |
| | | RLMSLAPSMATIYTDPIAAEKAAESWAKSTDTPADA |
| | | KGQPSGDASGSSYEEKSIPTELLQVGDIVVIRPGDKIP |
| | | ADGVVMRGETYVDESMVTGEAMPVQKRIGSNVIGG |
| | | TVNGNGRVDFRVTRAGRDTQLSQIVKLVQDAQTTR |
| | | APIQKVADTLAGYFVPTILLLGILTFLGWLILSHALSH |
| | | PPMIFLKNTSGGKVMICVKLCISVIVFACPCALGLATP |
| | | TAVMVGTGVGAENGILIKGGAALERTTQVTKVVLD |
| | | KTGTITRGKMEVAKSGLVFPWNDNVSQTKVWWAA |
| | | VGLAEMGSEHPIGRAILAAAKAEVGILEAEAAIPGSV |
| | | NDFKLTVGKGIDAIVEPALSGDRTRYRVLAGNVTFLE |
| | | ENGVEVPKDAVEAAERINSSVKSSRAKAVTAGTTNIF |
| | | VAIDGKYSGHLCLSDTIKDGAAGVISVLHSMGIKTA |
| | | MVTGDQRPTALAVAALVGISPEDVFAGVSPDQKQVI |
| | | VQQFQNQGEVVAMVGDGINDSPALATADVGIAMSS |
| | | GTDVAMEAADVVLMRPDDLLSIPSAIHLTRTIFRRIK |
| | | LNLAWACIYNIVGLPIAMGFFLPFGIHMHPMFAGFA |
| | | MACSSISVVVSSLALRWWQRPQWMDEASEPAGGLR |
| | | WMSGTGIVGWAKETFGRVRRGKREEGYVALENLEV |
| 7 | gene sequence of T. | ATGCTGTTGTCAGCGTCCCTCTCGGCGTTGGCCTTG |
| | reesei tyrosinase | GCCACAGTTTCACTCGCACAGGGCACGACACACAT |
| | | CCCCGTCACCGGTGTTCCCGTCTCTCCTGGTGCTGC |
| | | CGTGCCGCTGAGACAGAACATCAATGACCTGGCCA |
| | | AGTCCGGGCCGCAATGGTGAGTGACGCCCTCCTTC |
| | | CACCACACTTTACCTCAGTCAAGAGACAAGAGGG |
| | | AGACAAGTACAAAGCGGATGAAAAGAGGTGGACA |
| | | AGAGAGAGAGAGAGAGAAAGTGTGTGTGTGTATG |
| | | TGAGAGCGAGAGAGAGAGAGAGAGACAAGAGCT |
| | | ATTGGATGGACCAGGAGCCAGCATGGAGAACAGG |
| | | GGGAGACTTGACGATTCGAGGAGAGGGGGGCTCA |
| | | CATGTGCGTGCGAATAGGGATCTCTACGTTCAGGC |
| | | CATGTACAACATGTCCAAGATGGACTCCCATGACC |
| | | CGTACAGCTTCTTCCAGATTGCCGGTAAATATACA |
| | | TCTCGGCCTCCTGCGAGGCGACGTGACTCTCGGAG |
| | | CTTTTAGTAACACCAGCTAGGCATCCACGGCGCAC |
| | | CGTACATTGAGTACAACAAGGCCGGAGCAAAGTC |
| | | GGGCGATGGCTGGCTGGGCTACTGCCCTCACGGTG |
| | | TATGTGTTTTTGTCCATCGAGGAGGGCGCAAGAGT |
| | | TTCATGGACTTGAACTCTTCGCCCTTGTTGTGAGCC |
| | | GGAAATCATCGTCTCTGACAGTTTCATTAGGAGGA |
| | | CCTCTTCATCAGCTGGCACCGCCCCTATGTCCTGCT |
| | | CTTTGAGGTATGATTTGACCACGCTGGACTTTGAC |
| | | CTCATACAAACATCAACTGACATCGTTGCAGCAAG |
| | | CCTTGGTCTCCGTCGCCAAGGGCATCGCCAACTCG |
| | | TATCCCCCGTCTGTCCGCGCCAAGTACCAGGCTGC |
| | | CGCCGCCAGCCTGCGCGCCCCCTACTGGGACTGGG |
| | | CCGCCGACAGCTCCGTGCCCGCCGTCACCGTCCCC |
| | | CAGACGCTCAAGATCAACGTCCCCAGCGGCAGCA |
| | | GCACCAAGACCGTCGACTACACCAACCCGCTCAAG |
| | | ACGTACTACTTCCCGCGCATGTCCTTGACCGGCTC |
| | | GTACGGCGAGTTCACCGGCGGAGGCAACGACCAC |
| | | ACCGTCCGCTGCGCCGCCTCCAAGCAGAGCTATCC |
| | | CGCCACCGCCAACTCCAACCTGGCTGCCCGTCCTT |
| | | ACAAGTCCTGGATCGTACGTAGTCCCCCTTTCCCTT |
| | | TGGAAGCTTCCCCTTGAGTAAAGCTCGTCACTGAC |
| | | ACAGAGAGCGGCCCGCAGTACGATGTCCTGACCA |
| | | ACTCTCAAAACTTTGCCGACTTCGCCTCCACCAGC |
| | | GGCCCCGGCATCAACGTTGAGCAGATCCACAACGC |
| | | CATCCACTGGGACGGTGCTTGCGGCTCCCAGTTCC |
| | | TCGCCCCCGACTACTCCGGCTTCGACCCCCTGTTGT |
| | | AAGTCAATCGAGACGTCAAGAGTCATCTTGTCAAC |
| | | AACCGATGGCAAACGCAGTCTGTACTGACGCTGCA |
| | | AAATAGCTTCATGCACCACGCCCAGGTCGACCGCA |
| | | TGTGGGCCTTCTGGGAGGCCATCATGCCCTCGTCG |
| | | CCCCTCTTCACGGCCTCGTACAAGGGCCAGTCGCG |
| | | CTTCAACTCCAAGTCGGGCAGCACCATCACCCCCG |
| | | ACTCGCCCCTGCAGCCCTTCTACCAGGCCAACGGC |
| | | AAGTTCCACACGTCCAACACGGTCAAGAGCATCCA |
| | | GGGCATGGGCTACTCGTACCAGGGCATCGAGTACT |
| | | GGCAAAAGTCCCAGGCCCAGATCAAGTCGAGCGT |
| | | CACCACCATCATCAACCAGCTGTACGGGCCCAACT |
| | | CGGGCAAGAAGCGCAACGCCCCGCGCGACTTCTTG |
| | | AGCGACATTGTCACCGACGTCGAGAACCTCATCAA |
| | | GACCCGTTACTTTGCCAAGATCTCGGTCAACGTGA |
| | | CCGAGGTGACGGTCCGCCCCGCCGAGATCAACGTC |
| | | TACGTCGGCGGCCAGAAGGCCGGCAGCTTGATCGT |
| | | CATGAAGCTCCCCGCCGAGGGCACGGTCAACGGC |
| | | GGCTTCACCATTGACAACCCCATGCAAAGCATCCT |
| | | GCACGGTGGTCTCCGCAACGCCGTCCAGGCCTTTA |
| | | CCGAGGACATTGAGGTTGAGATTCTCTCTGTAAGT |
| | | TTTCCCCCCTCTCTCCACTCCCGACCACTCACTGTC |
| | | ACTATTTCGACTAGTCACCGTCAAGATGTGTATTT |
| | | GTTTGCTGACCCCCAAGCGCAGAAGGACGGACAA |
| | | GCCATCCCCCTCGAGACGGTCCCCAGCCTGTCCAT |
| | | CGACCTCGAGGTCGCCAACGTCACCCTGCCCTCCG |
| | | CCCTCGACCAGCTGCCCAAGTACGGCCAGCGCTCC |
| | | AGGCACCGCGCCAAGGCCGCCCAGCGCGGACACC |
| | | GCTTTGCCGTTCCCCATATCCCTCCTCTGTAA |
| 8 | coding sequence of | ATGCTGTTGTCAGCGTCCCTCTCGGCGTTGGCCTTG |
| | T. reesei tyrosinase | GCCACAGTTTCACTCGCACAGGGCACGACACACAT |
| | | CCCCGTCACCGGTGTTCCCGTCTCTCCTGGTGCTGC |
| | | CGTGCCGCTGAGACAGAACATCAATGACCTGGCCA |
| | | AGTCCGGGCCGCAATGGGATCTCTACGTTCAGGCC |
| | | ATGTACAACATGTCCAAGATGGACTCCCATGACCC |
| | | GTACAGCTTCTTCCAGATTGCCGGCATCCACGGCG |
| | | CACCGTACATTGAGTACAACAAGGCCGGAGCAAA |
| | | GTCGGGCGATGGCTGGCTGGGCTACTGCCCTCACG |
| | | GTGAGGACCTCTTCATCAGCTGGCACCGCCCCTAT |
| | | GTCCTGCTCTTTGAGCAAGCCTTGGTCTCCGTCGCC |
| | | AAGGGCATCGCCAACTCGTATCCCCCGTCTGTCCG |
| | | CGCCAAGTACCAGGCTGCCGCCGCCAGCCTGCGCG |
| | | CCCCCTACTGGGACTGGGCCGCCGACAGCTCCGTG |
| | | CCCGCCGTCACCGTCCCCCAGACGCTCAAGATCAA |
| | | CGTCCCCAGCGGCAGCAGCACCAAGACCGTCGACT |
| | | ACACCAACCCGCTCAAGACGTACTACTTCCCGCGC |
| | | ATGTCCTTGACCGGCTCGTACGGCGAGTTCACCGG |
| | | CGGAGGCAACGACCACACCGTCCGCTGCGCCGCCT |
| | | CCAAGCAGAGCTATCCCGCCACCGCCAACTCCAAC |
| | | CTGGCTGCCCGTCCTTACAAGTCCTGGATCTACGA |
| | | TGTCCTGACCAACTCTCAAAACTTTGCCGACTTCG |
| | | CCTCCACCAGCGGCCCCGGCATCAACGTTGAGCAG |
| | | ATCCACAACGCCATCCACTGGGACGGTGCTTGCGG |
| | | CTCCCAGTTCCTCGCCCCCGACTACTCCGGCTTCGA |
| | | CCCCCTGTTCTTCATGCACCACGCCCAGGTCGACC |
| | | GCATGTGGGCCTTCTGGGAGGCCATCATGCCCTCG |
| | | TCGCCCCTCTTCACGGCCTCGTACAAGGGCCAGTC |
| | | GCGCTTCAACTCCAAGTCGGGCAGCACCATCACCC |
| | | CCGACTCGCCCCTGCAGCCCTTCTACCAGGCCAAC |
| | | GGCAAGTTCCACACGTCCAACACGGTCAAGAGCAT |
| | | CCAGGGCATGGGCTACTCGTACCAGGGCATCGAGT |
| | | ACTGGCAAAAGTCCCAGGCCCAGATCAAGTCGAG |
| | | CGTCACCACCATCATCAACCAGCTGTACGGGCCCA |
| | | ACTCGGGCAAGAAGCGCAACGCCCCGCGCGACTT |
| | | CTTGAGCGACATTGTCACCGACGTCGAGAACCTCA |
| | | TCAAGACCCGTTACTTTGCCAAGATCTCGGTCAAC |
| | | GTGACCGAGGTGACGGTCCGCCCCGCCGAGATCA |
| | | ACGTCTACGTCGGCGGCCAGAAGGCCGGCAGCTTG |
| | | ATCGTCATGAAGCTCCCCGCCGAGGGCACGGTCAA |
| | | CGGCGGCTTCACCATTGACAACCCCATGCAAAGCA |
| | | TCCTGCACGGTGGTCTCCGCAACGCCGTCCAGGCC |
| | | TTTACCGAGGACATTGAGGTTGAGATTCTCTCTAA |
| | | GGACGGACAAGCCATCCCCCTCGAGACGGTCCCCA |
| | | GCCTGTCCATCGACCTCGAGGTCGCCAACGTCACC |
| | | CTGCCCTCCGCCCTCGACCAGCTGCCCAAGTACGG |
| | | CCAGCGCTCCAGGCACCGCGCCAAGGCCGCCCAG |
| | | CGCGGACACCGCTTTGCCGTTCCCCATATCCCTCCT |
| | | CTGTAA |
| 9 | amino acid | MLLSASLSALALATVSLAQGTTHIPVTGVPVSPGAAV |
| | sequence of T. | PLRQNINDLAKSGPQWDLYVQAMYNMSKMDSHDP |
| | reesei tyrosinase | YSFFQIAGIHGAPYIEYNKAGAKSGDGWLGYCPHGE |
| | (underlined is | DLFISWHRPYVLLFEQALVSVAKGIANSYPPSVRAKY |
| | signal peptide; | QAAAASLRAPYWDWAADSSVPAVTVPQTLKINVPS |
| | mature enzyme | GSSTKTVDYTNPLKTYYFPRMSLTGSYGEFTGGGND |
| | does not include | HTVRCAASKQSYPATANSNLAARPYKSWIYDVLTNS |
| | this underlined | QNFADFASTSGPGINVEQIHNAIHWDGACGSQFLAPD |
| | sequence) | YSGFDPLFFMHHAQVDRMWAFWEAIMPSSPLFTASY |
| | | KGQSRFNSKSGSTITPDSPLQPFYQANGKFHTSNTVK |
| | | SIQGMGYSYQGIEYWQKSQAQIKSSVTTIINQLYGPN |
| | | SGKKRNAPRDFLSDIVTDVENLIKTRYFAKISVNVTE |
| | | VTVRPAEINVYVGGQKAGSLIVMKLPAEGTVNGGFT |
| | | IDNPMQSILHGGLRNAVQAFTEDIEVEILSKDGQAIPL |
| | | ETVPSLSIDLEVANVTLPSALDQLPKYGQRSRHRAKA |
| | | AQRGHRFAVPHIPPL |
| 10 | mature amino acid | AIGPVADLHIVNKDLAPDGVQRPTVLAGGTFPGTLIT |
| | sequence of laccase | GQKGDNFQLNVIDDLTDDRMLTPTSIHWHGFFQKGT |
| | D from Cerrena | AWADGPAFVTQCPIIADNSFLYDFDVPDQAGTFWYH |
| | unicolor (mature = | SHLSTQYCDGLRGAFVVYDPNDPHKDLYDVDDGGT |
| | without signal | VITLADWYHVLAQTVVGAATPDSTLINGLGRSQTGP |
| | sequence) | ADAELAVISVEHNKRYRFRLVSISCDPNFTFSVDGHN |
| | | MTVIEVDGVNTRPLTVDSIQIFAGQRYSFVLNANQPE |
| | | DNYWIRAMPNIGRNTTTLDGKNAAILRYKNASVEEP |
| | | KTVGGPAQSPLNEADLRPLVPAPVPGNAVPGGADIN |
| | | HRLNLTFSNGLFSINNASFTNPSVPALLQILSGAQNAQ |
| | | DLLPTGSYIGLELGKVVELVIPPLAVGGPHPFHLHGH |
| | | NFWVVRSAGSDEYNFDDAILRDVVSIGAGTDEVTIRF |
| | | VTDNPGPWFLHCHIDWHLEAGLAIVFAEGINQTAAA |
| | | NPTPQAWDELCPKYNGLSASQKVKPKKGTAI |
| 11 | mature amino acid | HGHINDIVINGVWYQAYDPTTFPYESNPPIVVGWTA |
| | sequence of | ADLDNGFVSPDAYQNPDIICHKNATNAKGHASVKAG |
| | GH61A from T. | DTILFQWVPVPWPHPGPIVDYLANCNGDCETVDKTT |
| | reesei (mature = | LEFFKIDGVGLLSGGDPGTWASDVLISNNNTWVVKIP |
| | without signal | DNLAPGNYVLRHEIIALHSAGQANGAQNYPQCFNIA |
| | sequence) | VSGSGSLQPSGVLGTDLYHATDPGVLINIYTSPLNYII |
| | | PGPTVVSGLPTSVAQGSSAATATASATVPGGGSGPTS |
| | | RTTTTARTTQASSRPSSTPPATTSAPAGGPTQTLYGQ |
| | | CGGSGYSGPTRCAPPATCSTLNPYYAQCLN |
| 12 | Copper ion | MDMGDGSSQSCKISMLWNWYTVDACFLSSSWRIRN |
| | transmembrane | RGMFAASCIGIVLLVASVELMRRIGQEYDNSIVRQW |
| | transporter of T. | HRQAAMASDRAGGRTQGSASYCERLLFRATPLQQL |
| | reesei (website: | VRAIIHAATFGAAYIVMLLAMYFNGYIIICIIVGSGVG |
| | genome.jgi- | KFACHWLSVEIDLQPGEGERLLPKPILQTTICCD |
| | psf.org/Trire2/ | |
| | Trire2.home.html | |
| | protein ID: 52315) | |
| 13 | Copper ion | MLWNWNVMNTCFISKHWQITSKGMFAGSCIGVILLV |
| | transmembrane | IALEFLRRLSKEYDRFLIKQHAAPRAVPAFRPSVLQQ |
| | transporter of T. | ALRALLHVAQFSVAYIVMLLAMYYNGYFIICIFIGAYI |
| | reesei (website: | GSFVFHWEPLTAG |
| | genome.jgi- | |
| | psf.org/Trire2/ | |
| | Trire2.home.html | |
| | protein ID: 62716) | |
| 14 | Copper ion | MDHSHHMHAMEGHEGHGGHGGGMQDMCSMNMLF |
| | transmembrane | TWDTTNLCIVFRQWHVRSTASLIFSLIAVVLLGIGYE |
| | transporter of T. | ALRSVSRRYEASLATRLETVPRQNRETVSKRGHVIKA |
| | reesei (website: | TLYAIQNFYAFMLMLVFMTYNGWVMVAVSLGAFV |
| | genome.jgi- | GYLLFGHSTSATKDNACH |
| | psf.org/Trire2/ | |
| | Trire2.home.html | |
| | protein ID: 71029) | |
| 15 | Copper ion | MTMLMAMVFQTDIRTPLYANSWTPHHAGAYAGTCI |
| | transmembrane | FLIALAVIARLLVAFRARQERIWADHDARRRYVVVN |
| | transporter of T. | GKEPVAERLSRDSDAKSATMVISENGVEERVVVVEK |
| | reesei (website: | KDGATRPWRFSVDPVRAAMDTVIVGVGYLLMLAV |
| | genome.jgi- | MTMNVGYFMSVLGGTFLGSLLVGRYSEVYHH |
| | psf.org/Trire2/ | |
| | Trire2.home.html | |
| | protein ID: 108749) | |

1. A method for producing a cuproenzyme from a host cell comprising: overexpressing a copper metallochaperone in a host cell that expresses a cuproenzyme, and culturing the host cell under conditions sufficient to produce the cuproenzyme, wherein the host cell produces an increased amount of the cuproenzyme as compared to a corresponding host cell that does not overexpress the copper metallochaperone when cultured under substantially the same culture conditions.

2. The method of claim 1, wherein the cuproenzyme is secreted from the host cell.

3. The method of claim 1, wherein the cuproenzyme is selected from the group consisting of a lytic polysaccharide mono-oxygenase (LPMO), a laccase, a tyrosinase, an amine oxidase, a bilirubin oxidase, a catechol oxidase, a dopamine beta-monooxygenase, a galactose oxidase, a hexose oxidase, a L-ascorbate oxidase, a peptidylglycine monooxygenase, a polyphenol oxidase, a quercetin 2,3-dioxygenase, and a superoxide dismutase.

4. The method of claim 1, wherein the cuproenzyme is endogenous to the host cell.

5. The method of claim 1, wherein the cuproenzyme is heterologous to the host cell.

6. The method of claim 1, wherein the expression of the cuproenzyme and/or the copper metallochaperone is controlled by a promoter derived from the host cell.

7. The method of claim 6, wherein the host cell is a Trichoderma reesei (T reesei) cell and the promoter is a pyruvate kinase (pki) or cellobiohydrolase I (cbh1) promoter derived from T. reesei.

8. The method of claim 1, wherein the host cell expresses at least one additional cuproenzyme, wherein the production level of the at least one additional cuproenzyme is increased as compared to that of a corresponding host cell which does not overexpress the copper metallochaperone under substantially the same culture conditions.

9. The method of claim 1, wherein the copper matallochaperone is a membrane-bound copper transporting ATPase.

10. The method of claim 9, wherein the membrane-bound copper transporting ATPase comprises an amino acid sequence that is at least 60% identical to SEQ ID NO:6.

11-16. (canceled)

17. The method of claim 1, wherein the host cell is a filamentous fungal host cell.

18. The method of claim 17, wherein the filamentous fungal host is selected from the group consisting of: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

19. The method of claim 17, wherein the filamentous fungal host cell is a T. reesei, an Aspergillus niger, an Aspergillus oryzae, or a Talaromyces emersonii host cell.

20-24. (canceled)

25. A recombinant host cell comprising:
a first polynucleotide encoding a cuproenzyme, and a second polynucleotide encoding a copper metallochaperone, wherein the cuproenzyme is expressed in the host cell and the copper metallochaperone is over-expressed in the host cell, and wherein the level of expression of the cuproenzyme is increased in the host cell as compared to a corresponding host cell that does not overexpress the copper metallochaperone under substantially the same culture conditions.

26. (canceled)

27. The recombinant host cell of claim 25 or 26, wherein the cuproenzyme is selected from the group consisting of: a lytic polysaccharide mono-oxygenase (LPMO), a laccase, a tyrosinase, an amine oxidase, a bilirubin oxidase, a catechol oxidase, a dopamine beta-monooxygenase, a galactose oxidase, a hexose oxidase, a L-ascorbate oxidase, a peptidylglycine monooxygenase, a polyphenol oxidase, a quercetin 2,3-dioxygenase, and a superoxide dismutase.

28. The recombinant host cell of claim 27, wherein the cuproenzyme is selected from those listed in Table 3.

29-30. (canceled)

31. The recombinant host cell of claim 30, wherein host cell is T reesei and the promoter is a pki or a cbh1 promoter derived from T reesei.

32. The recombinant host cell of claim 25, wherein the second polynucleotide encodes a membrane-bound copper transporting ATPase comprising an amino acid sequence that is at least 60% identical to SEQ ID NO:6.

33-35. (canceled)

36. The recombinant host cell of claim 25, wherein the recombinant host cell is a filamentous fungal host cell.

37. The recombinant host cell of claim 36, wherein the filamentous fungal host is selected from the group consisting of: Aspergillus, Acremonium, Aureobasidium, Beauveria, Cephalosporium, Ceriporiopsis, Chaetomium paecilomyces, Chrysosporium, Claviceps, Cochiobolus, Cryptococcus, Cyathus, Endothia, Endothia mucor, Fusarium, Gilocladium, Humicola, Magnaporthe, Myceliophthora, Myrothecium, Mucor, Neurospora, Phanerochaete, Podospora, Paecilomyces, Penicillium, Pyricularia, Rhizomucor, Rhizopus, Schizophylum, Stagonospora, Talaromyces, Trichoderma, Thermomyces, Thermoascus, Thielavia, Tolypocladium, Trichophyton, Trametes, and Pleurotus.

38. The recombinant host cell of claim 36, wherein the filamentous fungal host cell is a T reesei, an Aspergillus niger, an Aspergillus oryzae, or a Talaromyces emersonii host cell.

39. (canceled)

40. A supernatant obtained from a culture of the recombinant host cell of claim 25.

41. A supernatant obtained using the method of claim 1.
